(19) United States
(12) Patent Application Publication
(10) Pub. No.: US 2026/0015672 A1
(43) Pub. Date: Jan. 15, 2026

(54) BIOMARKERS IN PANCREATIC CANCER

(71) Applicant: City of Hope, Duarte, CA (US)

(72) Inventor: Ajay Goel, Duarte, CA (US)

(21) Appl. No.: 18/871,592

(22) PCT Filed: Jun. 9, 2023
(86) PCT No.: PCT/US2023/024959

§ 371 Date: Dec. 4, 2024

Related U.S. Application Data
(60) Provisional application No. 63/351,212, filed on Jun. 10, 2022.
(60) Provisional application No. 63/359,270, filed on Jul. 8, 2022.

(51) Int. Cl. C12Q 1/6886; A61K 45/00

(52) U.S. Cl. CPC C12Q 1/6886; A61K 45/00; C12Q 2600/158; C12Q 2600/178

(57) ABSTRACT

Provided herein, inter alia, are methods of diagnosing, monitoring, detecting, or treating pancreatic cancer in a patient by detecting the expression levels of RNA biomarkers in a biological sample.

### CROSS-REFERENCE TO RELATED APPLICATIONS

This application claims the benefit of priority to U.S. Application No. 63/359,270 filed Jul. 8, 2022, and U.S. Application No. 63/351,212 filed Jun. 10, 2022, the disclosures of which are incorporated by reference herein in their entirety.

### STATEMENT AS TO RIGHTS TO INVENTIONS MADE UNDER FEDERALLY SPONSORED RESEARCH AND DEVELOPMENT

This invention was made with government support under CA214254 awarded by the National Institutes of Health. The government has certain rights in the invention.

### BACKGROUND

Pancreatic ductal adenocarcinoma (PDAC) is the most common malignancy of the pancreas. PDAC presents a substantial health problem with rising incidence and is predicted to become the second leading cause of cancer-associated mortality within the next decade in United States. As per the standard treatment strategies, surgical removal of the localized tumor offers the only potential curative option for this disease. Clinical findings in recent years have unequivocally established that surgery followed with modem adjuvant chemotherapy significantly improves patient outcomes, with a median overall survival ranging from 46 to 54 months in PDAC patients. Unfortunately, however, the patients who present with localized, resectable, and potentially curable tumors at initial diagnosis are only less than 15-20% of all cases, whereas the remainder have a more advanced unresectable or metastatic disease. This is reflected in the data that despite recent advances in treatment modalities, the 5-year survival rates in PDAC patients have essentially not improved significantly in the recent decades.

One of the themes that has emerged in the recent years is that earlier diagnosis of disease offers a promising opportunity for a timely intervention and subsequent improvement in survival of patients suffering from this fatal malignancy. In this context, to date, several blood-based biomarkers have been evaluated for their clinical usefulness for early diagnosis of PDAC. Serum carbohydrate antigen 19-9 (CA19-9) remains the most well-documented and widely used serum biomarker in patients with PDAC. Although CA19-9 is commonly used to monitor disease progression and therapeutic response, it lacks satisfactory sensitivity or specificity for screening and early detection of patients with PDAC. Importantly, 15-25% of patients with PDAC, including those at early-stages, often exhibit CA19-9 levels less than 37 U/ml which is considered as normal. Furthermore, 5-10% of the general population is Lewis antigen-negative with no or low secretion of CA19-9. In view of these clinical challenges in the art, the present disclosure is directed to the urgent need to develop robust alternate, preferably non-invasive, biomarkers for the early diagnosis of PDAC.

### BRIEF SUMMARY

Provided herein are methods of treating pancreatic cancer in a patient in need thereof comprising administering to the patient an effective amount of an anti-cancer agent, administering to the patient an effective amount of radiation therapy, administering to the patient image-based screening, surgically removing all or a portion of the pancreas of the patient, or a combination of two or more thereof; wherein a biological sample obtained from the patient comprises an elevated expression level, relative to a control, of an RNA; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof.

Provided herein are methods of treating pancreatic cancer in a patient in need thereof comprising: (i) detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof; and (ii) administering to the patient an effective amount of an anti-cancer agent, administering to the patient an effective amount of radiation therapy, administering to the patient image-based screening, surgically removing all or a portion of the pancreas of the patient, or a combination of two or more thereof. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof.

Provided herein are methods of diagnosing a patient with pancreatic cancer comprising detecting an elevated expression level, relative to a control, of an RNA in a biological sample obtained from the patient, thereby diagnosing the patient with pancreatic cancer; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof.

These and other embodiments of the disclosure are described herein.

### BRIEF DESCRIPTION OF THE DRAWINGS

FIGS. 1A-1D: Expression level of identified cell-free and exosomal miRNA candidates for the diagnosis of patients with early-stages of PDAC obtained from genome-wide small RNA sequencing. FIGS. 1A-1B: Expression level of candidate cf-miRNAs and representative heatmap in patients with early-stage of PDAC (Stage I-II) versus non-disease control samples. FIGS. 1C-1D: Expression level of candidate exo-miRNAs and representative heatmap in patients with PDAC (Stage I-II) versus non-disease control samples. The miRNA expression profile was z-normalized. (miRNA: micro RNA; CPM: counts per million; FC: fold change; PDAC: pancreatic ductal adenocarcinoma (light blue) and Non-disease controls (dark blue)).

FIGS. 2A-2D: Performance evaluation of cell-free and exosomal miRNA biomarker panel in clinical cohorts by qRT-PCR. Representative heatmap of statistically significant and upregulated candidate FIG. 2A: cf-miRNAs and FIG. 2B: exo-miRNAs in patients with PDAC versus non-disease controls. FIG. 2C: ROC curves analysis for the cf-miRNA, exo-miRNA or cf- and exo-combination panel in the training cohort. FIG. 2D: ROC curves analysis for the cf-miRNA, exo-miRNA and cf and exosomal combination panels in the validation cohort. (Exo: exosomal; miRNA: micro RNA; qRT-PCR: Quantitative Reverse transcription polymerase chain reaction; cf: cell-free; AUC: Area under the curve; ROC: receiver operating characteristic)

FIGS. 3A-3D: Prioritization and performance evaluation of cell-free and exosomal miRNA biomarker panel in clinical cohorts. FIG. 3A: ROC curve analysis for the cf-miRNA, exo-miRNA or cf and exosomal combination panel in the training cohort. FIG. 3B: ROC curve analysis for the cf-miRNA, exo-miRNA or cf and exosomal combination panels in the validation cohort. FIG. 3C: ROC curve analysis to identify early-stages (stage I and II) and late stages (stage III and IV) PDAC patients from non-disease controls in validation cohort. FIG. 3D: Risk score analysis in all stages PDAC patients and non-disease controls in the validation cohort. ROC curves are shown with 95% CIs. (*p<0.001, Exo: exosomal; miRNA: micro RNA; cf: cell-free; AUC: Area under the curve; TNM: tumor-node-metastasis; PDAC: pancreatic ductal adenocarcinoma; ROC: receiver operating characteristic)

FIGS. 4A-4E: Performance evaluation of the miRNA signature in combination with CA19-9, and diagnostic potential evaluation by decision curve analysis and calibration curve analysis. FIG. 4A: ROC analysis to compare diagnostic performances between cf and exosomal combination miRNA signature and CA19-9 in all stages of PDAC patients. FIG. 4B: ROC analysis to compare diagnostic performance between cf and exosomal combination miRNA signature and CA19-9 in early-stages (Stage I and II) of PDAC patients. FIG. 4C: Performance of cf and exosomal combination miRNA signature in the cohort of 81 participant (22 PDAC and 59 non-disease controls) who presented with al CA19-9 level less than 37 U/mL. FIG. 4D: Decision curve analysis and FIG. 4E: Calibration curve analysis to evaluate the performance of the combined miRNA biomarker panel. ROC curves are shown with 95% CTs. (miRNA: micro RNA; CA19-9: carbohydrate antigen 19-9; AUC: Area under the curve; Exo: exosomal; cf: cell-free; ROC: receiver operating characteristic; PDAC: pancreatic ductal adenocarcinoma)

FIG. 5 provides a summary of the diagnostic performance of the cell-free and exosomal miRNA-based biomarker panel in the training and validation cohorts.

FIG. 6 provides the diagnostic performance of CA19-9 alone and miRNA signature in combination with CA19-9 after fixing their specificity at 95% and 99%.

FIG. 7 shows the clinicopathological characteristics of pancreatic ductal adenocarcinoma patients and non-disease controls.

FIG. 8 provides a summary of the diagnostic performance of the cell free and exosomal miRNA-based biomarkers.

FIG. 9 shows the miRNA regulatory using hypergeometric tests on cancer hallmarks and KEGG pathways. Significantly enriched signaling pathways (BH-adjusted P<0.001) were illustrated in the dot plot.

FIG. 10 provides a schematic flow chart highlighting intended use of exosome-based miRNA signature for screening of PDAC.

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

As used herein, the term “tumor-derived exosome” or “exosome” refers to a small (between 20-300 nm in diameter) vesicle comprising a lipid bilayer membrane that encloses an internal space, and which is generated from a cancer cell by direct plasma membrane budding or by fusion of the late endosome with the plasma membrane. The components of tumor-derived exosomes include proteins, DNA, mRNA, microRNA, long noncoding RNA, circular RNA, and the like, which play a role in regulating tumor growth, metastasis, and angiogenesis in the process of cancer development.

“Exosomal RNA” refers to RNA within a tumor-derived exosome or RNA obtained from within a tumor-derived exosome. In embodiments, “exosomal RNA” is exosomal miRNA. In embodiments, “exosomal RNA” is exosomal mRNA. Exosomal RNA can be detected and measured by methods known in the art, such as those described herein.

“Cell-free RNA” or “cf-RNA” refers to RNA that is not within a tumor-derived exosome or RNA that has not been obtained from within a tumor-derived exosome. Cell-free RNA can be detected and measured by methods known in the art, such as those described herein.

A “cell” refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian (e.g. human) cells. Cells may be useful when they are naturally nonadherent or have been treated not to adhere to surfaces, for example by trypsinization.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides, contemplated herein include any types of RNA, e.g. mRNA, siRNA, miRNA, and guide RNA and any types of DNA, genomic DNA. plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

A “microRNA,” “microRNA nucleic acid sequence,” “miR,” “miRNA” as used herein, refers to a nucleic acid that functions in RNA silencing and post-transcriptional regulation of gene expression. The term includes all forms of a miRNA, such as the pri-, pre-, and mature forms of the miRNA. In embodiments, microRNAs (miRNAs) are short (20-24 nt) non-coding RNAs that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. The mature miRNA is incorporated into a RNA-induced silencing complex (RISC), which recognizes target mRNAs through imperfect base pairing with the miRNA and most commonly results in translational inhibition or destabilization of the target mRNA.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding RNA that is present within the cell or the amount of protein encoded by that DNA produced by the cell. The level of expression of non-coding nucleic acid molecules (e.g., miRNA, mRNA) may be detected by standard PCR or Northern blot methods well known in the art. See, Sambrook et al., 1989 Molecular Cloning: A Laboratory Manual, 18.1-18.88.

The terms “expression level,” “amount,” or “level” of a biomarker is a detectable level in a biological sample. “Expression” generally refers to the process by which information (e.g., gene-encoded and/or epigenetic) is converted into the structures present and operating in the cell. Therefore, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, miRNA, transfer RNA, ribosomal RNA, non-coding RNA). Expression levels can be measured by methods known to one skilled in the art and also disclosed herein. The expression level or amount of a biomarker (e.g., RNA, miRNA) can be used to diagnose and/or treat a subject with pancreatic cancer.

The terms an “elevated expression level” or “elevated level” of gene expression is an expression level of the gene that is higher than the expression level of the gene in a control. The control may be any suitable control, as described herein. In embodiments, an “elevated expression level” of the biomarker gene compared to the control (when the expression level of the biomarker is greater than the corresponding control) is, for example, an increase in the expression level of about 10%, 20%, 30%, 40%, 50%, 60%, 70%, 80%, 90%, 95%, 96%, 97%, 98% or 99% or greater relative to the control. In embodiments, an “elevated expression level” of the biomarker gene is an amount that is statistically significantly greater than the expression level of the control.

The terms “does not have an elevated expression level” or an expression level that is “not elevated” is an expression level of the gene that is about the same as (or lower than) the expression level of the gene in a control. The control may be any suitable control, as described herein. In embodiments, “about the same as” is +/−25% of the expression level of the biomarker gene in a control. In embodiments, “about the same as” is +/−20% of the expression level of the biomarker gene in a control. In embodiments, “about the same as” is +/−15% of the expression level of the biomarker gene in a control. In embodiments, “about the same as” is +/−10% of the expression level of the biomarker gene in a control. In embodiments, “about the same as” is +/−5% of the expression level of the biomarker gene in a control. In embodiments, an expression level of the biomarker gene that is “not elevated” or “unelevated” is an amount that is not statistically significantly different than the expression level of the control.

The terms “biomarker gene” and “biomarker” are used interchangeably and in accordance with their plain and ordinary meaning. In embodiments, a biomarker is a gene or a set of genes (i.e., a biomarker gene). Biomarkers include, but are not limited to, polynucleotides (e.g., DNA, and/or RNA), polynucleotide copy number alterations (e.g., DNA copy numbers), polypeptides, or polypeptide and polynucleotide modifications (e.g., posttranslational modifications). In embodiments, a biomarker refers to RNA (e.g., miRNA), the expression level of which is associated with a particular biological state, particularly a state associated with pancreatic cancer.

Biomarker levels may be detected at either the protein or gene expression level. Proteins expressed by biomarkers can be quantified by immunohistochemistry (IHC) or flow cytometry with an antibody that detects the proteins. Biomarker expression can be quantified by multiple platforms such as real-time polymerase chain reaction (rtPCR), Nanostring, RNAseq, or in situ hybridization. There is a range of biomarker expression across as measured by Nanostring. In embodiments, quantitative rtPCR, Nanostring, RNAseq, and in situ hybridization are platforms to quantitate biomarker gene expression. For Nanostring, RNA is extracted from a biological sample and a known quantity of RNA is placed on the Nanostring machine for gene expression detection using gene specific probes. The number of counts of biomarkers within a sample is determined and normalized to a set of housekeeping genes. To determine a threshold for increased or decreased biomarker levels, one skilled in the art could assess biomarker levels in a control group of samples and select the 10^{th}, 20^{th}, 25^{th}, 30^{th}, 40^{th}, 50^{th}, 60^{th}, 70^{th}, 75^{th}, 80^{th} or 90^{th} percentile of biomarker gene expression. In embodiments, the increased or decreased expression of biomarkers may be determined by calculating the H-score for the expression of the biomarkers. Thus, the increased or decreased expression of biomarkers may have an H-score. As used herein, an “H-score” or “Histoscore” is a numerical value determined by a semi-quantitative method commonly known for immunohistochemically evaluating protein expression in tumor samples. The H-score may be calculated using the following formula: [1×(% cells 1+)+2×(% cells 2+)+3×(% cells 3+)]. According to this formula, the H-score is calculated by determining the percentage of cells having a given staining intensity level (i.e., level 1+, 2+, or 3+ from lowest to highest intensity level), weighting the percentage of cells having the given intensity level by multiplying the cell percentage by a factor (e.g., 1, 2, or 3) that gives more relative weight to cells with higher-intensity membrane staining, and summing the results to obtain a H-score. Commonly H-scores range from 0 to 300. Further description on the determination of H-scores in tumor cells can be found in Hirsch et al, J Clin Oncol 21: 3798-3807, 2003 and John et al, Oncogene 28:S14-S23, 2009. IHC or other methods known in the art may be used for detecting biomarker expression.

“Control” is used in accordance with its plain ordinary meaning and refers to an assay, comparison, or experiment in which the subjects or reagents of the experiment are treated as in a parallel experiment except for omission of a procedure, reagent, or variable of the experiment. In embodiments, the control is used as a standard of comparison in evaluating experimental effects. In embodiments, a control is the measurement of the activity or level of RNA. In embodiments, a control is a healthy patient or a healthy population of patients. In embodiments, a control is an average value from a population of similar patients, e.g., healthy patients with a similar medical background, age, weight, etc. In embodiments, the control is a healthy patient or a population of healthy patients. In embodiments, a healthy patient can be referred to as a non-diseased patient or non-diseased control. In embodiments, the control is a population of non-diseased patients. In embodiments, a non-diseased patient is a patient that does not have cancer. In embodiments, a non-diseased patient is a patient that does not have pancreatic cancer. In embodiments, the control is a patient that does not have cancer or a population of patients that do not have cancer. In embodiments, the control is a patient that does not have pancreatic cancer or a population of patients that do not have pancreatic cancer. In embodiments, the control is a patient that does not have PDAC or a population of patients that do not have PDAC. In embodiments, the control is an average value from population of healthy patients. A control can also be obtained from the same patient, e.g., from an earlier-obtained sample, prior to disease, or prior to treatment. One of skill will recognize that controls can be designed for assessment of any number of parameters. In embodiments, a control is a negative control. In embodiments, such as some embodiments relating to detecting the level of expression of a gene/protein or a subset of genes/proteins, a control comprises the average amount of expression (e.g., protein or mRNA) in a population of subjects (e.g., with cancer) or in a healthy or general population. In embodiments, the control comprises an average amount (e.g. amount of expression) in a population in which the number of subjects (n) is 5 or more, 20 or more, 50 or more, 100 or more, 1,000 or more, and the like. In embodiments, the control is a standard control. In embodiments, a standard control is a level of expression of the biomarker (e.g., RNA, miRNA) that has been correlated with the diagnosis of pancreatic cancer in a subject. In embodiments, a standard control is a level of expression of the biomarker (e.g., RNA, miRNA) that has been correlated with a healthy subject (i.e., a subject that does not have pancreatic cancer). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “healthy patient” refers to a non-diseased patient. In embodiments, a healthy patient is a patient that does not have cancer. In embodiments, a healthy patient is a patient that does not have pancreatic cancer (e.g., PDAC).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid including two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein including two or more subsequences that are not found in the same relationship to each other in nature (e.g., a fusion protein).

The phrase “specifically (or selectively) binds” to an antibody or “specifically (or selectively) immunoreactive with,” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The terms “isolate” or “isolated”, when applied to a nucleic acid, virus, or protein, denotes that the nucleic acid, virus, or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. An RNA that is the predominant species present in a preparation is substantially purified.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (e.g., www.ncbi.nlm.nih.gov/BLAST/or the like). Such sequences are then the to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

As used herein, the term “about” means a range of values including the specified value, which a person of ordinary skill in the art would consider reasonably similar to the specified value. In embodiments, “about” means within a standard deviation using measurements generally acceptable in the art. In embodiments, “about” means a range extending to +/−10% of the specified value. In embodiments, “about” includes the specified value.

The singular terms “a,” “an,” and “the” include the plural reference unless the context clearly indicates otherwise.

A “therapeutic agent” or “anticancer agent” as used herein refer to an agent (e.g., compound, pharmaceutical composition) that when administered to a subject will have the intended therapeutic effect, e.g., treatment or amelioration of pancreatic cancer, or their symptoms including any objective or subjective parameter of treatment such as abatement; remission; diminishing of symptoms or making the cancer more tolerable to the patient; slowing in the rate of degeneration or decline; making the final point of degeneration less debilitating; or improving a patient's physical or mental well-being.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. In embodiments, a biological sample is blood. In embodiments, a biological sample is a serum sample (e.g., the fluid and solute component of blood without the clotting factors). In embodiments, a biological sample is a plasma sample (e.g, the liquid portion of blood). In embodiments, a biological sample is cell-free RNA obtained from blood. In embodiments, a biological sample is an exosome obtained from a blood sample, wherein the exosome comprises RNA. In embodiments, a biological sample is an exosome obtained from a serum sample, wherein the exosome comprises RNA. In embodiments, a biological sample is an exosome obtained from a plasma sample, wherein the exosome comprises RNA.

“Liquid biological sample” refers to liquid materials obtained or derived from a subject or patient. Liquid biological samples include bodily fluids such as blood and blood fractions or products (e.g., serum, plasma, platelets, red blood cells, and the like), sputum, urine, synovial fluid, and the like. In embodiments, a liquid biological sample is a blood sample.

The term “diagnosis” is used in accordance with its plain and ordinary meaning and refers to an identification or likelihood of the presence of a disease (e.g., pancreatic cancer) or outcome in a subject.

“Image-based screening” refers to methods using imaging technology to detect a cancer or tumor in a patient. Exemplary types of image-based screening include x-rays, computed tomography (CT), magnetic resonance imaging (MRI), positron emission tomography (PET), and ultrasound. In embodiments of the methods described herein, the image-based screening is CT, MRI, or ultrasound. In embodiments, the ultrasound is endoscopic ultrasonography (EUS). In embodiments of the methods described herein, the image-based screening is CT, MRI, or EUS. In embodiments of the methods described herein, the image-based screening is MRI or EUS. In embodiments of the methods described herein, the image-based screening is CT. In embodiments of the methods described herein, the image-based screening is MRI. In embodiments of the methods described herein, the image-based screening is EUS.

The terms “treating” or “treatment” are used in accordance with their plain and ordinary meaning and broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, delay or slowing of disease progression, amelioration or palliation of the disease state, and remission, whether partial or total and whether detectable or undetectable. Treatment may inhibit the disease's spread; relieve the disease's symptoms, fully or partially remove the disease's underlying cause, shorten a disease's duration, or do a combination of these things. Treatment methods include administering to a subject a therapeutically effective amount of an active agent. The term “treating” does not including preventing.

The term “preventing” or “prevent” is used in accordance with its plain and ordinary meaning and refers to a decrease in the occurrence of disease symptoms in a patient or to keep a disease from occurring. The prevention may be complete (no detectable symptoms) or partial, such that fewer symptoms are observed than would likely occur absent treatment.

An “effective amount” is an amount sufficient to accomplish a stated purpose (e.g. achieve the effect for which it is administered, treat a disease). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques. In embodiments, “therapeutically effective amount” refers to the amount of the therapeutic agent sufficient to treat or ameliorate pancreatic cancer, as described above. For any therapeutic agent described herein, the therapeutically effective amount can be initially determined from cell culture assays. Target concentrations will be those concentrations of active compound(s) that are capable of achieving the methods described herein, as measured using the methods described herein or known in the art. As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. Dosages may be varied depending upon the requirements of the patient and the therapeutic agent being employed. The dose administered to a patient should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the patient's disease state. A “therapeutically effective amount” can also be found on the label or Prescribing Information for commercially available therapeutic agents.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

The terms “patient” or “subject” are used in accordance with its plain and ordinary meaning and refer to a living organism suffering from or prone to a disease that can be treated by administration of a pharmaceutical composition, such as anti-cancer agents and chemotherapeutic agents. Non-limiting examples include humans, other mammals, bovines, rats, mice, dogs, cats, monkeys, and other non-mammalian animals. In embodiments, a patient is human. In embodiments, the human patient is at least 45 years old. In embodiments, the human patient is at least 50 years old. In embodiments, the human patient is at least 55 years old. In embodiments, the human patient is at least 60 years old. In embodiments, the human patient is at least 45 years old and has new-onset diabetes. In embodiments, the human patient is at least 50 years old and has new-onset diabetes. In embodiments, the human patient is at least 55 years old and has new-onset diabetes. In embodiments, the human patient has a family history of pancreatic cancer. In embodiments, the human patient has obesity. In embodiments, the human patient has a history of pancreatitis. In embodiments, the human patient has a history of chronic pancreatitis.

The terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., pancreas, which site is referred to as a primary tumor, e.g., primary pancreatic cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if pancreatic cancer metastasizes to the lymph nodes, the secondary tumor at the site of the lymph nodes consist of pancreatic cancer cells and not abnormal lymph node cells. The secondary tumor in the lymph nodes is referred to as lymph node metastasis. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors.

“PDAC” refers to pancreatic ductal adenocarcinoma, a type of pancreatic cancer. PDAC accounts for more than 90% of cases of pancreatic cancer. Symptoms of pancreatic cancer include diabetes, hyperglycemia, jaundice, sudden weight loss, abdominal pain, back pain, persistent loss of appetite, light-colored stools, bloating, nausea, vomiting, or diarrhea. In embodiments, a symptom of pancreatic cancer is new-onset diabetes. In embodiments, a symptom of pancreatic cancer is new-onset hyperglycemia. In embodiments, a symptom of pancreatic cancer is diabetes. In embodiments, a symptom of pancreatic cancer is hyperglycemia. In embodiments, diabetes is new-onset diabetes or pre-existing diabetes.

Stages of pancreatic cancer are well known in the art. For example, “Stage 1” pancreatic cancer refers to pancreatic cancer that is only found in the pancreas. Stage 1 encompasses Stages IA and IB, wherein Stage IA is defined as T1, N0, M0, and Stage IB is defined as T2, N0, M0). “Stage 2” pancreatic cancer refers to pancreatic cancer may have metastasized to nearby tissue and organs or lymph nodes near the pancreas. Stage 2 pancreatic cancer encompasses Stages IIA and IIB, wherein Stage IIA is defined as T3, N0, M0, and Stage IIB is defined as T1/T2/T3, N1, M0. “Stage 3” pancreatic cancer refers to pancreatic cancer that has spread to the major blood vessels near the pancreas and may have spread to nearby lymph nodes, but not to distant sites (T4, any N, M0). “Stage 4” pancreatic cancer refers to pancreatic cancer that may be of any size and have spread to distant organs (e.g., liver, lung, peritoneal cavity) and may have spread to lymph nodes or organs and tissues near the pancreas (any T, any N, M1). The term “T1” means the tumor is only in the pancreas and is 2 cm or smaller in size. The term “T2” means the tumor is only in the pancreas and is larger than 2 cm and smaller than 4 cm. The term “T3” means the tumor is larger than 4 cm and that extends beyond the pancreas, but does not involve major arteries or veins near the pancreas. The term “T4” means the tumor extends beyond the pancreas into major arteries or veins near the pancreas. The term “N0” means the cancer was not found in the regional lymph nodes. The term “N1” means the cancer has spread to 1-3 regional lymph nodes. The term “N2” means the cancer has spread to 4 or more regional lymph nodes. The term “M0” means the cancer has not spread to other parts of the body. The term “M1” means the cancer has spread to another part of the body, including distant lymph nodes.

The term “carbohydrate antigen 19-9” or “CA19-9” refers to serum CA19-9 which is the most well-documented and widely used serum biomarker in patients with PDAC (Refs 2, 3, 12). Although CA19-9 is commonly used to monitor disease progression and therapeutic response in pancreatic cancer, it lacks satisfactory sensitivity or specificity for screening and early detection of patients with PDAC. (Refs 13-14). 15-25% of patients with PDAC, including those at early-stages (e.g., Stage 1 and 2), often exhibit CA19-9 levels less than 37 U/ml which is considered as normal. (Refs 15-16). Furthermore, 5-10% of the general population is Lewis antigen-negative with no or low secretion of CA19-9 (Ref 17). The phrase “a normal level of CA19-9” refers to CA19-9 levels less than 37 U/ml. The phrase “an elevated level of CA19-9” refers to CA19-9 levels of 37 U/ml or higher. The phrase “a rising carbohydrate antigen 19-9 level” refers to a level of CA19-9 taken in a patient that is elevated when compared to the level of CA19-9 taken at an earlier point in time. “A rising carbohydrate antigen 19-9 level” can be greater than or less than 37 U/ml at any time point.

### Methods of Treatment

Provided herein are methods of treating pancreatic cancer in a patient in need thereof, the method comprising administering to the patient an effective amount of an anti-cancer agent, administering to the patient an effective amount of radiation therapy, administering to the patient image-based screening, surgically removing all or a portion of the pancreas of the patient, or a combination of two or more thereof; wherein a biological sample obtained from the patient comprises an elevated expression level, relative to a control, of an RNA; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof. In embodiments, the RNA comprises at least two RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eight RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least nine RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least ten RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eleven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least twelve RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eight RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises nine RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises ten RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eleven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises twelve RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the pancreatic cancer is Stage 3 pancreatic cancer or Stage 4 pancreatic cancer. In embodiments, the pancreatic cancer is Stage 3 pancreatic cancer. In embodiments, the pancreatic cancer is Stage 4 pancreatic cancer. In embodiments, the pancreatic cancer is Stage 1 pancreatic cancer, Stage 2 pancreatic cancer, or Stage 3 pancreatic cancer. In embodiments, the pancreatic cancer is Stage 1 pancreatic cancer, Stage 2 pancreatic cancer, Stage 3 pancreatic cancer, or Stage 4 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent. In embodiments, the method comprises administering to the patient an effective amount of an anti-cancer agent and administering to the patient an effective amount of radiation therapy. In embodiments, the method comprises administering to the patient an effective amount of an anti-cancer agent and surgically removing all or a portion of the pancreas of the patient, or a combination of two or more thereof. In embodiments, the method comprises administering to the patient an effective amount of radiation therapy. In embodiments, the method comprises surgically removing all or a portion of the pancreas of the patient. In embodiments, the method comprises administering to the patient an effective amount of an anti-cancer agent, administering to the patient an effective amount of radiation therapy, and surgically removing all or a portion of the pancreas of the patient. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the biological sample obtained from the patient further comprises an elevated expression level, relative to a control, of an RNA selected from the group consisting of cell-free let-7e-5p, cell-free miR-26a-5p, cell-free miR-223-3p, cell-free miR-340-3p, cell-free let-7f-5p, cell-free miR-369-3p, cell-free miR-125a-5p, cell-free miR-495-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-375-3p, exosomal miR-199a-5p, and a combination of two or more thereof. In embodiments, the biological sample obtained from the patient further comprises an elevated expression level, relative to a control, of an RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, and a combination of two or more thereof. In embodiments, the biological sample obtained from the patient further comprises an elevated expression level, relative to a control, of an RNA selected from the group consisting of cell-free let-7f-5p, cell-free miR-369-3p, cell-free miR-125a-5p, cell-free miR-495-3p, exosomal miR-375-3p, exosomal miR-199a-5p, and a combination of two or more thereof.

Provided herein are methods of treating pancreatic cancer in a patient in need thereof, the method comprising administering to the patient an effective amount of an anti-cancer agent, administering to the patient an effective amount of radiation therapy, surgically removing all or a portion of the pancreas of the patient, or a combination of two or more thereof; wherein a biological sample obtained from the patient comprises an elevated expression level, relative to a control, of an RNA; wherein the RNA comprises cell-free miR-30c-5p, cell-free miR-142-3p, cell-free miR-340-5p, cell-free miR-335-5p, cell-free miR-23b-3p, or a combination of two or more thereof. In embodiments, the RNA comprises at least two RNA selected from the group consisting of cell-free miR-30c-5p, cell-free miR-142-3p, cell-free miR-340-5p, cell-free miR-335-5p, and cell-free miR-23b-3p. In embodiments, the RNA comprises at least three RNA selected from the group consisting of cell-free miR-30c-5p, cell-free miR-142-3p, cell-free miR-340-5p, cell-free miR-335-5p, and cell-free miR-23b-3p. In embodiments, the RNA comprises at least four RNA selected from the group consisting of cell-free miR-30c-5p, cell-free miR-142-3p, cell-free miR-340-5p, cell-free miR-335-5p, and cell-free miR-23b-3p. In embodiments, the RNA comprises cell-free miR-30c-5p, cell-free miR-142-3p, cell-free miR-340-5p, cell-free miR-335-5p, and cell-free miR-23b-3p. In embodiments, the RNA comprises two RNA selected from the group consisting of cell-free miR-30c-5p, cell-free miR-142-3p, cell-free miR-340-5p, cell-free miR-335-5p, and cell-free miR-23b-3p. In embodiments, the RNA comprises three RNA selected from the group consisting of cell-free miR-30c-5p, cell-free miR-142-3p, cell-free miR-340-5p, cell-free miR-335-5p, and cell-free miR-23b-3p. In embodiments, the RNA comprises four RNA selected from the group consisting of cell-free miR-30c-5p, cell-free miR-142-3p, cell-free miR-340-5p, cell-free miR-335-5p, and cell-free miR-23b-3p. In embodiments, the RNA comprises cell-free miR-30c-5p, cell-free miR-142-3p, cell-free miR-340-5p, cell-free miR-335-5p, and cell-free miR-23b-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the pancreatic cancer is Stage 3 pancreatic cancer or Stage 4 pancreatic cancer. In embodiments, the pancreatic cancer is Stage 3 pancreatic cancer. In embodiments, the pancreatic cancer is Stage 4 pancreatic cancer. In embodiments, the pancreatic cancer is Stage 1 pancreatic cancer, Stage 2 pancreatic cancer, or Stage 3 pancreatic cancer. In embodiments, the pancreatic cancer is Stage 1 pancreatic cancer, Stage 2 pancreatic cancer, Stage 3 pancreatic cancer, or Stage 4 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent. In embodiments, the method comprises administering to the patient an effective amount of an anti-cancer agent and administering to the patient an effective amount of radiation therapy. In embodiments, the method comprises administering to the patient an effective amount of an anti-cancer agent and surgically removing all or a portion of the pancreas of the patient, or a combination of two or more thereof. In embodiments, the method comprises administering to the patient an effective amount of radiation therapy. In embodiments, the method comprises surgically removing all or a portion of the pancreas of the patient. In embodiments, the method comprises administering to the patient an effective amount of an anti-cancer agent, administering to the patient an effective amount of radiation therapy, and surgically removing all or a portion of the pancreas of the patient. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the biological sample obtained from the patient further comprises an elevated expression level, relative to a control, of an RNA selected from the group consisting of cell-free let-7e-5p, cell-free miR-26a-5p, cell-free miR-223-3p, cell-free miR-340-3p, cell-free let-7f-5p, cell-free miR-369-3p, cell-free miR-125a-5p, cell-free miR-495-3p, and a combination of two or more thereof. In embodiments, the biological sample obtained from the patient further comprises an elevated expression level, relative to a control, of an RNA selected from the group consisting of cell-free let-7e-5p, cell-free miR-26a-5p, cell-free miR-223-3p, cell-free miR-340-3p, and a combination of two or more thereof. In embodiments, the biological sample obtained from the patient further comprises an elevated expression level, relative to a control, of an RNA selected from the group consisting of cell-free let-7f-5p, cell-free miR-369-3p, cell-free miR-125a-5p, cell-free miR-495-3p, and a combination of two or more thereof.

Provided herein are methods of treating pancreatic cancer in a patient in need thereof, the method comprising administering to the patient an effective amount of an anti-cancer agent, administering to the patient an effective amount of radiation therapy, surgically removing all or a portion of the pancreas of the patient, or a combination of two or more thereof; wherein a biological sample obtained from the patient comprises an elevated expression level, relative to a control, of an RNA; wherein the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof. In embodiments, the RNA comprises at least two RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the pancreatic cancer is Stage 3 pancreatic cancer or Stage 4 pancreatic cancer. In embodiments, the pancreatic cancer is Stage 3 pancreatic cancer. In embodiments, the pancreatic cancer is Stage 4 pancreatic cancer. In embodiments, the pancreatic cancer is Stage 1 pancreatic cancer, Stage 2 pancreatic cancer, or Stage 3 pancreatic cancer. In embodiments, the pancreatic cancer is Stage 1 pancreatic cancer, Stage 2 pancreatic cancer, Stage 3 pancreatic cancer, or Stage 4 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent. In embodiments, the method comprises administering to the patient an effective amount of an anti-cancer agent and administering to the patient an effective amount of radiation therapy. In embodiments, the method comprises administering to the patient an effective amount of an anti-cancer agent and surgically removing all or a portion of the pancreas of the patient, or a combination of two or more thereof. In embodiments, the method comprises administering to the patient an effective amount of radiation therapy. In embodiments, the method comprises surgically removing all or a portion of the pancreas of the patient. In embodiments, the method comprises administering to the patient an effective amount of an anti-cancer agent, administering to the patient an effective amount of radiation therapy, and surgically removing all or a portion of the pancreas of the patient. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the biological sample obtained from the patient further comprises an elevated expression level, relative to a control, of an RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-375-3p, exosomal miR-199a-5p, and a combination of two or more thereof. In embodiments, the biological sample obtained from the patient further comprises an elevated expression level, relative to a control, of an RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, and a combination of two or more thereof. In embodiments, the biological sample obtained from the patient further comprises an elevated expression level, relative to a control, of an RNA selected from the group consisting of exosomal miR-375-3p, exosomal miR-199a-5p, and a combination of two or more thereof.

Provided herein are methods of treating pancreatic cancer in a patient in need thereof comprising administering to the patient an effective amount of an anti-cancer agent, administering to the patient an effective amount of radiation therapy, surgically removing all or a portion of the pancreas of the patient, or a combination of two or more thereof; wherein a biological sample obtained from the patient comprises an elevated expression level, relative to a control, of an RNA; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof. In embodiments, the elevated expression level of the RNA indicates that the patient has pancreatic cancer. In embodiments, the pancreatic cancer is pancreatic ductal adenocarcinoma. In embodiments, the pancreatic cancer is Stage 1 pancreatic cancer or Stage 2 pancreatic cancer. In embodiments, the biological sample obtained from the patient has a normal level of CA19-9. In embodiments, the RNA comprises cell-free miR-30c-5p, cell-free miR-142-3p, cell-free miR-340-5p, cell-free miR-335-5p, cell-free miR-23b-3p, cell-free let-7e-5p, cell-free miR-26a-5p, cell-free miR-223-3p, cell-free miR-340-3p, cell-free let-7f-5p, cell-free miR-369-3p, cell-free miR-125a-5p, cell-free miR-495-3p, or a combination of two or more thereof. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-375-3p, exosomal miR-199a-5p, or a combination of two or more thereof. In embodiments, the control is the average expression level of RNA in a population of healthy patients.

Provided herein are methods of treating pancreatic cancer in a patient in need thereof comprising administering to the patient an effective amount of an anti-cancer agent, administering to the patient an effective amount of radiation therapy, surgically removing all or a portion of the pancreas of the patient, or a combination of two or more thereof; wherein a biological sample obtained from the patient comprises an elevated expression level, relative to a control, of an RNA; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof. In embodiments, the RNA comprises at least two RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eight RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least nine RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least ten RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eleven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least twelve RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least thirteen RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eight RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises nine RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises ten RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eleven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises twelve RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises thirteen RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent, surgically removing all or a portion of the pancreas of a patient or a combination thereof.

Provided herein are methods of treating pancreatic cancer in a patient in need thereof, the method comprising administering to the patient an effective amount of an anti-cancer agent, administering to the patient an effective amount of radiation therapy, surgically removing all or a portion of the pancreas of the patient, or a combination of two or more thereof; wherein a biological sample obtained from the patient comprises an elevated expression level, relative to a control, of an RNA; wherein the RNA comprises cell-free miR-30c-5p, cell-free miR-142-3p, cell-free miR-340-5p, cell-free miR-335-5p, cell-free miR-23b-3p, cell-free let-7e-5p, cell-free miR-26a-5p, cell-free miR-223-3p, cell-free miR-340-3p, or a combination of two or more thereof. In embodiments, the RNA comprises at least two RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises at least three RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises at least four RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises at least five RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises two RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises three RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises four RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises five RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage 2 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the method comprises administering to the patient an effective amount of an anti-cancer agent, surgically removing all or a portion of the pancreas of a patient, or a combination thereof. In embodiments, the control is the average expression level of RNA in a population of healthy patients.

Provided herein are methods of treating pancreatic cancer in a patient in need thereof, the method comprising administering to the patient an effective amount of an anti-cancer agent, administering to the patient an effective amount of radiation therapy, surgically removing all or a portion of the pancreas of the patient, or a combination of two or more thereof; wherein a biological sample obtained from the patient comprises an elevated expression level, relative to a control, of an RNA; wherein the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof. In embodiments, the RNA comprises at least two RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent, surgically removing all or a portion of the pancreas of the patient, or a combination thereof.

### Methods of Detecting and Treating

Provided herein are methods of treating pancreatic cancer in a patient in need thereof, the method comprising: (i) detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof; and (ii) administering to the patient an effective amount of an anti-cancer agent, administering to the patient an effective amount of radiation therapy, surgically removing all or a portion of the pancreas of the patient, or a combination of two or more thereof. The elevated expression level of the RNA, relative to a control, indicates that the patient has pancreatic cancer. Upon diagnosing the patient with pancreatic cancer, treatment is initiated by administering to the patient an effective amount of an anti-cancer agent, administering to the patient an effective amount of radiation therapy, surgically removing all or a portion of the pancreas of the patient, or a combination of two or more thereof. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the RNA comprises at least two RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eight RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least nine RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least ten RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eleven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least twelve RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eight RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises nine RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises ten RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eleven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises twelve RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the pancreatic cancer is Stage 3 pancreatic cancer or Stage 4 pancreatic cancer. In embodiments, the pancreatic cancer is Stage 3 pancreatic cancer. In embodiments, the pancreatic cancer is Stage 4 pancreatic cancer. In embodiments, the pancreatic cancer is Stage 1 pancreatic cancer, Stage 2 pancreatic cancer, or Stage 3 pancreatic cancer. In embodiments, the pancreatic cancer is Stage 1 pancreatic cancer, Stage 2 pancreatic cancer, Stage 3 pancreatic cancer, or Stage 4 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent. In embodiments, the method comprises administering to the patient an effective amount of an anti-cancer agent and administering to the patient an effective amount of radiation therapy. In embodiments, the method comprises administering to the patient an effective amount of an anti-cancer agent and surgically removing all or a portion of the pancreas of the patient, or a combination of two or more thereof. In embodiments, the method comprises administering to the patient an effective amount of radiation therapy. In embodiments, the method comprises surgically removing all or a portion of the pancreas of the patient. In embodiments, the method comprises administering to the patient an effective amount of an anti-cancer agent, administering to the patient an effective amount of radiation therapy, and surgically removing all or a portion of the pancreas of the patient. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the biological sample obtained from the patient further comprises an elevated expression level, relative to a control, of an RNA selected from the group consisting of cell-free let-7e-5p, cell-free miR-26a-5p, cell-free miR-223-3p, cell-free miR-340-3p, cell-free let-7f-5p, cell-free miR-369-3p, cell-free miR-125a-5p, cell-free miR-495-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-375-3p, exosomal miR-199a-5p, and a combination of two or more thereof. In embodiments, the biological sample obtained from the patient further comprises an elevated expression level, relative to a control, of an RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, and a combination of two or more thereof. In embodiments, the biological sample obtained from the patient further comprises an elevated expression level, relative to a control, of an RNA selected from the group consisting of cell-free let-7f-5p, cell-free miR-369-3p, cell-free miR-125a-5p, cell-free miR-495-3p, exosomal miR-375-3p, exosomal miR-199a-5p, and a combination of two or more thereof.

Provided herein are methods of treating pancreatic cancer in a patient in need thereof, the method comprising: (i) detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, or a combination of two or more thereof; and (ii) administering to the patient an effective amount of an anti-cancer agent, administering to the patient an effective amount of radiation therapy, surgically removing all or a portion of the pancreas of the patient, or a combination of two or more thereof. The elevated expression level of the RNA, relative to a control, indicates that the patient has pancreatic cancer. Upon diagnosing the patient with pancreatic cancer, treatment is initiated by administering to the patient an effective amount of an anti-cancer agent, administering to the patient an effective amount of radiation therapy, surgically removing all or a portion of the pancreas of the patient, or a combination of two or more thereof. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the RNA comprises at least two RNA selected from the group consisting of cell-free miR-30c-5p, cell-free miR-142-3p, cell-free miR-340-5p, cell-free miR-335-5p, and cell-free miR-23b-3p. In embodiments, the RNA comprises at least three RNA selected from the group consisting of cell-free miR-30c-5p, cell-free miR-142-3p, cell-free miR-340-5p, cell-free miR-335-5p, and cell-free miR-23b-3p. In embodiments, the RNA comprises at least four RNA selected from the group consisting of cell-free miR-30c-5p, cell-free miR-142-3p, cell-free miR-340-5p, cell-free miR-335-5p, and cell-free miR-23b-3p. In embodiments, the RNA comprises cell-free miR-30c-5p, cell-free miR-142-3p, cell-free miR-340-5p, cell-free miR-335-5p, and cell-free miR-23b-3p. In embodiments, the RNA comprises two RNA selected from the group consisting of cell-free miR-30c-5p, cell-free miR-142-3p, cell-free miR-340-5p, cell-free miR-335-5p, and cell-free miR-23b-3p. In embodiments, the RNA comprises three RNA selected from the group consisting of cell-free miR-30c-5p, cell-free miR-142-3p, cell-free miR-340-5p, cell-free miR-335-5p, and cell-free miR-23b-3p. In embodiments, the RNA comprises four RNA selected from the group consisting of cell-free miR-30c-5p, cell-free miR-142-3p, cell-free miR-340-5p, cell-free miR-335-5p, and cell-free miR-23b-3p. In embodiments, the RNA comprises cell-free miR-30c-5p, cell-free miR-142-3p, cell-free miR-340-5p, cell-free miR-335-5p, and cell-free miR-23b-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the pancreatic cancer is Stage 3 pancreatic cancer or Stage 4 pancreatic cancer. In embodiments, the pancreatic cancer is Stage 3 pancreatic cancer. In embodiments, the pancreatic cancer is Stage 4 pancreatic cancer. In embodiments, the pancreatic cancer is Stage 1 pancreatic cancer, Stage 2 pancreatic cancer, or Stage 3 pancreatic cancer. In embodiments, the pancreatic cancer is Stage 1 pancreatic cancer, Stage 2 pancreatic cancer, Stage 3 pancreatic cancer, or Stage 4 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent. In embodiments, the method comprises administering to the patient an effective amount of an anti-cancer agent and administering to the patient an effective amount of radiation therapy. In embodiments, the method comprises administering to the patient an effective amount of an anti-cancer agent and surgically removing all or a portion of the pancreas of the patient, or a combination of two or more thereof. In embodiments, the method comprises administering to the patient an effective amount of radiation therapy. In embodiments, the method comprises surgically removing all or a portion of the pancreas of the patient. In embodiments, the method comprises administering to the patient an effective amount of an anti-cancer agent, administering to the patient an effective amount of radiation therapy, and surgically removing all or a portion of the pancreas of the patient. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the method further comprises detecting an elevated expression level, relative to a control, of an RNA selected from the group consisting of cell-free let-7e-5p, cell-free miR-26a-5p, cell-free miR-223-3p, cell-free miR-340-3p, cell-free let-7f-5p, cell-free miR-369-3p, cell-free miR-125a-5p, cell-free miR-495-3p, and a combination of two or more thereof. In embodiments, the method further comprises detecting an elevated expression level, relative to a control, of an RNA selected from the group consisting of cell-free let-7e-5p, cell-free miR-26a-5p, cell-free miR-223-3p, cell-free miR-340-3p, and a combination of two or more thereof. In embodiments, the method further comprises detecting an elevated expression level, relative to a control, of an RNA selected from the group consisting of cell-free let-7f-5p, cell-free miR-369-3p, cell-free miR-125a-5p, cell-free miR-495-3p, and a combination of two or more thereof.

Provided herein are methods of treating pancreatic cancer in a patient in need thereof, the method comprising: (i) detecting an elevated expression level, relative to a control, of an RNA in a biological sample obtained from the patient, wherein the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof; and (ii) administering to the patient an effective amount of an anti-cancer agent, administering to the patient an effective amount of radiation therapy, surgically removing all or a portion of the pancreas of the patient, or a combination of two or more thereof. The elevated expression level of the RNA, relative to a control, indicates that the patient has pancreatic cancer. Upon diagnosing the patient with pancreatic cancer, treatment is initiated by administering to the patient an effective amount of an anti-cancer agent, administering to the patient an effective amount of radiation therapy, surgically removing all or a portion of the pancreas of the patient, or a combination of two or more thereof. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the RNA comprises at least two RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the pancreatic cancer is Stage 3 pancreatic cancer or Stage 4 pancreatic cancer. In embodiments, the pancreatic cancer is Stage 3 pancreatic cancer. In embodiments, the pancreatic cancer is Stage 4 pancreatic cancer. In embodiments, the pancreatic cancer is Stage 1 pancreatic cancer, Stage 2 pancreatic cancer, or Stage 3 pancreatic cancer. In embodiments, the pancreatic cancer is Stage 1 pancreatic cancer, Stage 2 pancreatic cancer, Stage 3 pancreatic cancer, or Stage 4 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent. In embodiments, the method comprises administering to the patient an effective amount of an anti-cancer agent and administering to the patient an effective amount of radiation therapy. In embodiments, the method comprises administering to the patient an effective amount of an anti-cancer agent and surgically removing all or a portion of the pancreas of the patient, or a combination of two or more thereof. In embodiments, the method comprises administering to the patient an effective amount of radiation therapy. In embodiments, the method comprises surgically removing all or a portion of the pancreas of the patient. In embodiments, the method comprises administering to the patient an effective amount of an anti-cancer agent, administering to the patient an effective amount of radiation therapy, and surgically removing all or a portion of the pancreas of the patient. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the method further comprises detecting an elevated expression level, relative to a control, of an RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-375-3p, exosomal miR-199a-5p, and a combination of two or more thereof. In embodiments, the method further comprises detecting an elevated expression level, relative to a control, of an RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, and a combination of two or more thereof. In embodiments, the method further comprises detecting an elevated expression level, relative to a control, of an RNA selected from the group consisting of exosomal miR-375-3p, exosomal miR-199a-5p, and a combination of two or more thereof.

Provided herein are methods of treating pancreatic cancer in a patient in need thereof comprising: (i) detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof, and (ii) administering to the patient an effective amount of an anti-cancer agent, administering to the patient an effective amount of radiation therapy, surgically removing all or a portion of the pancreas of the patient, or a combination of two or more thereof. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the elevated expression level of the RNA indicates that the patient has pancreatic cancer. In embodiments, the pancreatic cancer is pancreatic ductal adenocarcinoma. In embodiments, the pancreatic cancer is Stage 1 pancreatic cancer or Stage 2 pancreatic cancer. In embodiments, the biological sample obtained from the patient has a normal level of CA19-9. In embodiments, the RNA comprises cell-free miR-30c-5p, cell-free miR-142-3p, cell-free miR-340-5p, cell-free miR-335-5p, cell-free miR-23b-3p, cell-free let-7e-5p, cell-free miR-26a-5p, cell-free miR-223-3p, cell-free miR-340-3p, cell-free let-7f-5p, cell-free miR-369-3p, cell-free miR-125a-5p, cell-free miR-495-3p, or a combination of two or more thereof. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-375-3p, exosomal miR-199a-5p, or a combination of two or more thereof. In embodiments, the control is the average expression level of RNA in a population of healthy patients.

Provided herein are methods of treating pancreatic cancer in a patient in need thereof comprising: (i) detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof; and (ii) administering to the patient an effective amount of an anti-cancer agent, administering to the patient an effective amount of radiation therapy, surgically removing all or a portion of the pancreas of the patient, or a combination of two or more thereof. In embodiments, the RNA comprises at least two RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eight RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least nine RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least ten RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eleven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least twelve RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least thirteen RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eight RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises nine RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises ten RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eleven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises twelve RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises thirteen RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage 1113. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent, surgically removing all or a portion of the pancreas of the patient, or a combination thereof.

Provided herein are methods of treating pancreatic cancer in a patient in need thereof comprising. (i) detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, cell-free let-7e-5p, cell-free miR-26a-5p, cell-free miR-223-3p, cell-free miR-340-3p, or a combination of two or more thereof; and (ii) administering to the patient an effective amount of an anti-cancer agent, administering to the patient an effective amount of radiation therapy, surgically removing all or a portion of the pancreas of the patient, or a combination of two or more thereof. In embodiments, the RNA comprises at least two RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises at least three RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises at least four RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises at least five RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises two RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises three RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises four RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises five RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage 2 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent, surgically removing all or a portion of the pancreas of the patient, or a combination thereof.

Provided herein are methods of treating pancreatic cancer in a patient in need thereof comprising: (i) detecting an elevated expression level, relative to a control, of an RNA in a biological sample obtained from the patient, wherein the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof; and (ii) administering to the patient an effective amount of an anti-cancer agent, administering to the patient an effective amount of radiation therapy, surgically removing all or a portion of the pancreas of the patient, or a combination of two or more thereof. In embodiments, the RNA comprises at least two RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent, surgically removing all or a portion of the pancreas of the patient, or a combination thereof.

### Methods of Diagnosis

Provided herein are methods of diagnosing a patient with pancreatic cancer comprising detecting an elevated expression level, relative to a control, of an RNA in a biological sample obtained from the patient, thereby diagnosing the patient with pancreatic cancer; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof. The elevated expression level of the RNA, relative to a control, indicates that the patient has pancreatic cancer. In embodiments, The elevated expression level of the RNA, relative to a control, indicates that the patient is likely to have pancreatic cancer. Upon diagnosing the patient with pancreatic cancer, treatment is initiated by administering to the patient an effective amount of an anti-cancer agent, administering to the patient an effective amount of radiation therapy, surgically removing all or a portion of the pancreas of the patient, or a combination of two or more thereof. In embodiments, the RNA comprises at least two RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eight RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least nine RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least ten RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eleven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least twelve RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eight RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises nine RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises ten RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eleven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises twelve RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the pancreatic cancer is Stage 3 pancreatic cancer or Stage 4 pancreatic cancer. In embodiments, the pancreatic cancer is Stage 3 pancreatic cancer. In embodiments, the pancreatic cancer is Stage 4 pancreatic cancer. In embodiments, the pancreatic cancer is Stage 1 pancreatic cancer, Stage 2 pancreatic cancer, or Stage 3 pancreatic cancer. In embodiments, the pancreatic cancer is Stage 1 pancreatic cancer, Stage 2 pancreatic cancer, Stage 3 pancreatic cancer, or Stage 4 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent. In embodiments, the method comprises administering to the patient an effective amount of an anti-cancer agent and administering to the patient an effective amount of radiation therapy. In embodiments, the method comprises administering to the patient an effective amount of an anti-cancer agent and surgically removing all or a portion of the pancreas of the patient, or a combination of two or more thereof. In embodiments, the method comprises administering to the patient an effective amount of radiation therapy. In embodiments, the method comprises surgically removing all or a portion of the pancreas of the patient. In embodiments, the method comprises administering to the patient an effective amount of an anti-cancer agent, administering to the patient an effective amount of radiation therapy, and surgically removing all or a portion of the pancreas of the patient. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the method further comprises detecting an elevated expression level, relative to a control, of an RNA selected from the group consisting of cell-free let-7e-5p, cell-free miR-26a-5p, cell-free miR-223-3p, cell-free miR-340-3p, cell-free let-7f-5p, cell-free miR-369-3p, cell-free miR-125a-5p, cell-free miR-495-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-375-3p, exosomal miR-199a-5p, and a combination of two or more thereof. In embodiments, the method further comprises detecting an elevated expression level, relative to a control, of an RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, and a combination of two or more thereof. In embodiments, the method further comprises detecting an elevated expression level, relative to a control, of an RNA selected from the group consisting of cell-free let-7f-5p, cell-free miR-369-3p, cell-free miR-125a-5p, cell-free miR-495-3p, exosomal miR-375-3p, exosomal miR-199a-5p, and a combination of two or more thereof.

Provided herein are methods of diagnosing a patient with pancreatic cancer comprising detecting an elevated expression level, relative to a control, of an RNA in a biological sample obtained from the patient, thereby diagnosing the patient with pancreatic cancer; wherein the RNA comprises cell-free miR-30c-5p, cell-free miR-142-3p, cell-free miR-340-5p, cell-free miR-335-5p, cell-free miR-23b-3p, or a combination of two or more thereof. The elevated expression level of the RNA, relative to a control, indicates that the patient has pancreatic cancer. In embodiments, The elevated expression level of the RNA, relative to a control, indicates that the patient is likely to have pancreatic cancer. The elevated expression level of the RNA, relative to a control, indicates that the patient has pancreatic cancer. In embodiments, the elevated expression level of the RNA, relative to a control, indicates that the patient likely has pancreatic cancer. Upon diagnosing the patient with pancreatic cancer, treatment is initiated by administering to the patient an effective amount of an anti-cancer agent, administering to the patient an effective amount of radiation therapy, surgically removing all or a portion of the pancreas of the patient, or a combination of two or more thereof. In embodiments, the RNA comprises at least two RNA selected from the group consisting of cell-free miR-30c-5p, cell-free miR-142-3p, cell-free miR-340-5p, cell-free miR-335-5p, and cell-free miR-23b-3p. In embodiments, the RNA comprises at least three RNA selected from the group consisting of cell-free miR-30c-5p, cell-free miR-142-3p, cell-free miR-340-5p, cell-free miR-335-5p, and cell-free miR-23b-3p. In embodiments, the RNA comprises at least four RNA selected from the group consisting of cell-free miR-30c-5p, cell-free miR-142-3p, cell-free miR-340-5p, cell-free miR-335-5p, and cell-free miR-23b-3p. In embodiments, the RNA comprises cell-free miR-30c-5p, cell-free miR-142-3p, cell-free miR-340-5p, cell-free miR-335-5p, and cell-free miR-23b-3p. In embodiments, the RNA comprises two RNA selected from the group consisting of cell-free miR-30c-5p, cell-free miR-142-3p, cell-free miR-340-5p, cell-free miR-335-5p, and cell-free miR-23b-3p. In embodiments, the RNA comprises three RNA selected from the group consisting of cell-free miR-30c-5p, cell-free miR-142-3p, cell-free miR-340-5p, cell-free miR-335-5p, and cell-free miR-23b-3p. In embodiments, the RNA comprises four RNA selected from the group consisting of cell-free miR-30c-5p, cell-free miR-142-3p, cell-free miR-340-5p, cell-free miR-335-5p, and cell-free miR-23b-3p. In embodiments, the RNA comprises cell-free miR-30c-5p, cell-free miR-142-3p, cell-free miR-340-5p, cell-free miR-335-5p, and cell-free miR-23b-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the pancreatic cancer is Stage 3 pancreatic cancer or Stage 4 pancreatic cancer. In embodiments, the pancreatic cancer is Stage 3 pancreatic cancer. In embodiments, the pancreatic cancer is Stage 4 pancreatic cancer. In embodiments, the pancreatic cancer is Stage 1 pancreatic cancer, Stage 2 pancreatic cancer, or Stage 3 pancreatic cancer. In embodiments, the pancreatic cancer is Stage 1 pancreatic cancer, Stage 2 pancreatic cancer, Stage 3 pancreatic cancer, or Stage 4 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method further comprises administering to the patient an effective amount of an anti-cancer agent. In embodiments, the method further comprises administering to the patient an effective amount of an anti-cancer agent and administering to the patient an effective amount of radiation therapy. In embodiments, the method further comprises administering to the patient an effective amount of an anti-cancer agent and surgically removing all or a portion of the pancreas of the patient, or a combination of two or more thereof. In embodiments, the method further comprises administering to the patient an effective amount of radiation therapy. In embodiments, the method further comprises surgically removing all or a portion of the pancreas of the patient, or a combination of two or more thereof. In embodiments, the method further comprises administering to the patient an effective amount of an anti-cancer agent, administering to the patient an effective amount of radiation therapy, and surgically removing all or a portion of the pancreas of the patient. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the method further comprises detecting an elevated expression level, relative to a control, of an RNA selected from the group consisting of cell-free let-7e-5p, cell-free miR-26a-5p, cell-free miR-223-3p, cell-free miR-340-3p, cell-free let-7f-5p, cell-free miR-369-3p, cell-free miR-125a-5p, cell-free miR-495-3p, and a combination of two or more thereof. In embodiments, the method further comprises detecting an elevated expression level, relative to a control, of an RNA selected from the group consisting of cell-free let-7e-5p, cell-free miR-26a-5p, cell-free miR-223-3p, cell-free miR-340-3p, and a combination of two or more thereof. In embodiments, the method further comprises detecting an elevated expression level, relative to a control, of an RNA selected from the group consisting of cell-free let-7f-5p, cell-free miR-369-3p, cell-free miR-125a-5p, cell-free miR-495-3p, and a combination of two or more thereof.

Provided herein are methods of diagnosing a patient with pancreatic cancer comprising detecting an elevated expression level, relative to a control, of an RNA in a biological sample obtained from the patient, thereby diagnosing the patient with pancreatic cancer; wherein the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof. The elevated expression level of the RNA, relative to a control, indicates that the patient has pancreatic cancer. In embodiments, The elevated expression level of the RNA, relative to a control, indicates that the patient is likely to have pancreatic cancer. Upon diagnosing the patient with pancreatic cancer, treatment can be initiated by administering to the patient an effective amount of an anti-cancer agent, administering to the patient an effective amount of radiation therapy, surgically removing all or a portion of the pancreas of the patient, or a combination of two or more thereof. In embodiments, the RNA comprises at least two RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the pancreatic cancer is Stage 3 pancreatic cancer or Stage 4 pancreatic cancer. In embodiments, the pancreatic cancer is Stage 3 pancreatic cancer. In embodiments, the pancreatic cancer is Stage 4 pancreatic cancer. In embodiments, the pancreatic cancer is Stage 1 pancreatic cancer, Stage 2 pancreatic cancer, or Stage 3 pancreatic cancer. In embodiments, the pancreatic cancer is Stage 1 pancreatic cancer, Stage 2 pancreatic cancer, Stage 3 pancreatic cancer, or Stage 4 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method further comprises administering to the patient an effective amount of an anti-cancer agent. In embodiments, the method further comprises administering to the patient an effective amount of an anti-cancer agent and administering to the patient an effective amount of radiation therapy. In embodiments, the method further comprises administering to the patient an effective amount of an anti-cancer agent and surgically removing all or a portion of the pancreas of the patient, or a combination of two or more thereof. In embodiments, the method further comprises administering to the patient an effective amount of radiation therapy. In embodiments, the method comprises surgically removing all or a portion of the pancreas of the patient. In embodiments, the method further comprises administering to the patient an effective amount of an anti-cancer agent, administering to the patient an effective amount of radiation therapy, and surgically removing all or a portion of the pancreas of the patient. In embodiments, the method further comprises detecting an elevated expression level, relative to a control, of an RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-375-3p, exosomal miR-199a-5p, and a combination of two or more thereof. In embodiments, the method further comprises detecting an elevated expression level, relative to a control, of an RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, and a combination of two or more thereof. In embodiments, the method further comprises detecting an elevated expression level, relative to a control, of an RNA selected from the group consisting of exosomal miR-375-3p, exosomal miR-199a-5p, and a combination of two or more thereof.

Provided herein are methods of diagnosing a patient with pancreatic cancer comprising detecting an elevated expression level, relative to a control, of an RNA in a biological sample obtained from the patient, thereby diagnosing the patient with pancreatic cancer; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof. In embodiments, the elevated expression level of the RNA indicates that the patient has pancreatic cancer. In embodiments, the methods further comprise administering to the patient an effective amount of an anti-cancer agent, administering to the patient an effective amount of radiation therapy, surgically removing all or a portion of the pancreas of the patient, or a combination of two or more thereof. In embodiments, the methods further comprise administering to the patient an effective amount of an anti-cancer agent. In embodiments, the pancreatic cancer is pancreatic ductal adenocarcinoma. In embodiments, the pancreatic cancer is Stage 1 pancreatic cancer or Stage 2 pancreatic cancer. In embodiments, the biological sample obtained from the patient has a normal level of CA19-9. In embodiments, the RNA comprises cell-free miR-30c-5p, cell-free miR-142-3p, cell-free miR-340-5p, cell-free miR-335-5p, cell-free miR-23b-3p, cell-free let-7e-5p, cell-free miR-26a-5p, cell-free miR-223-3p, cell-free miR-340-3p, cell-free let-7f-5p, cell-free miR-369-3p, cell-free miR-125a-5p, cell-free miR-495-3p, or a combination of two or more thereof. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-375-3p, exosomal miR-199a-5p, or a combination of two or more thereof. In embodiments, the control is the average expression level of RNA in a population of healthy patients.

Provided herein are methods of diagnosing a patient with pancreatic cancer comprising detecting an elevated expression level, relative to a control, of an RNA in a biological sample obtained from the patient, thereby diagnosing the patient with pancreatic cancer; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof. In embodiments, the RNA comprises at least two RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eight RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least nine RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least ten RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eleven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least twelve RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least thirteen RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miIR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eight RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises nine RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises ten RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eleven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises twelve RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises thirteen RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage LIB. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent, surgically removing all or a portion of the pancreas of the patient, or a combination thereof.

Provided herein are methods of diagnosing a patient with pancreatic cancer comprising detecting an elevated expression level, relative to a control, of an RNA in a biological sample obtained from the patient, thereby diagnosing the patient with pancreatic cancer; wherein the RNA comprises cell-free miR-30c-5p, cell-free miR-142-3p, cell-free miR-340-5p, cell-free miR-335-5p, cell-free miR-23b-3p, cell-free let-7e-5p, cell-free miR-26a-5p, cell-free miR-223-3p, cell-free miR-340-3p, or a combination of two or more thereof. In embodiments, the RNA comprises at least two RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises at least three RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises at least four RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises at least five RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises two RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises three RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises four RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises five RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage 2 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent, surgically removing all or a portion of the pancreas of the patient, or a combination thereof.

Provided herein are methods of diagnosing a patient with pancreatic cancer comprising detecting an elevated expression level, relative to a control, of an RNA in a biological sample obtained from the patient, thereby diagnosing the patient with pancreatic cancer; wherein the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof. In embodiments, the RNA comprises at least two RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent, surgically removing all or a portion of the pancreas of the patient, or a combination thereof.

### Methods of Monitoring

Provided herein are methods of monitoring a patient at risk for developing pancreatic cancer comprising: (i) detecting an expression level of an RNA in a biological sample obtained from the patient at a first point in time; (ii) detecting an expression level of an RNA in a biological sample obtained from the patient at a second point in time, wherein the second point in time is later than the first point in time; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof. Provided herein are methods of monitoring a patient at risk for developing pancreatic cancer comprising: (i) detecting an expression level of an RNA in a biological sample obtained from the patient at a first point in time; (ii) detecting an expression level of an RNA in a biological sample obtained from the patient at a second point in time, wherein the second point in time is later than the first point in time; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof. Provided herein are methods of monitoring a patient at risk for developing pancreatic cancer comprising: (i) detecting an expression level of an RNA in a biological sample obtained from the patient at a first point in time; (ii) detecting an expression level of an RNA in a biological sample obtained from the patient at a second point in time, wherein the second point in time is later than the first point in time; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof. In embodiments, the expression level of RNA at the second point in time is elevated when compared to the expression level of RNA at the first point in time. In embodiments, an elevated expression level of RNA at the second point in time when compared to the expression level of RNA at the first point in time indicates that the patient has an increased risk of developing pancreatic cancer or that the patient has developed pancreatic cancer. In embodiments, the expression level of RNA at the second point in time is about the same as the expression level of RNA at the first point in time. In embodiments, an expression level of RNA at the second point in time that is about the same as the expression level of RNA at the first point in time indicates that the patient does not have an increased risk of developing pancreatic cancer. In embodiments, the second point in time is about 6 months after the first point in time. In embodiments, the second point in time is about 1 year after the first point in time. In embodiments, the second point in time is about 18 months after the first point in time. In embodiments, the second point in time is about 2 years after the first point in time. In embodiments, the method of monitoring the patient is repeated once every 6 months, once per year, once every 18 months, once every two years, once every three years, once every four years, or once every five years. In embodiments, the patient is at risk for developing pancreatic cancer. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient is at risk for developing pancreatic cancer. In embodiments, the patient has diabetes. In embodiments, the patient has new-onset diabetes. In embodiments, the patient has pre-existing diabetes. In embodiments, the diabetes is Type 2 diabetes. In embodiments, the patient has obesity. In embodiments, the patient has a rising carbohydrate antigen 19-9 level. In embodiments, the patient has a family history of pancreatic cancer. In embodiments, the patient has a history of pancreatitis. In embodiments, the patient is 40 years old. In embodiments, the patient smokes cigarettes. In embodiments, the patient has a history of smoking cigarettes. In embodiments, the RNA comprises at least two RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eight RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least nine RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least ten RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eleven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least twelve RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eight RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises nine RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises ten RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eleven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises twelve RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method further comprises administering to the patient an effective amount of an anti-cancer agent, administering to the patient an effective amount of radiation therapy, surgically removing all or a portion of the pancreas of the patient, or a combination of two or more thereof.

### Methods of Detection

Provided herein are methods of detecting an RNA biomarker in a patient having, or suspected of having, pancreatic cancer comprising detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof; and wherein the RNA comprises the RNA biomarker. In embodiments, the patient has pancreatic cancer. In embodiments, the patient is suspected of having pancreatic cancer. The elevated expression level of the RNA, relative to a control, indicates that the patient has pancreatic cancer. In embodiments, The elevated expression level of the RNA, relative to a control, indicates that the patient is likely to have pancreatic cancer. In embodiments, the method further comprises administering to the patient an effective amount of an anti-cancer agent, administering to the patient an effective amount of radiation therapy, surgically removing all or a portion of the pancreas of the patient, or a combination of two or more thereof. In embodiments, the RNA comprises at least two RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eight RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least nine RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least ten RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eleven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least twelve RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eight RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises nine RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises ten RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eleven RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises twelve RNA selected from the group consisting of cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the pancreatic cancer is Stage 3 pancreatic cancer or Stage 4 pancreatic cancer. In embodiments, the pancreatic cancer is Stage 3 pancreatic cancer. In embodiments, the pancreatic cancer is Stage 4 pancreatic cancer. In embodiments, the pancreatic cancer is Stage 1 pancreatic cancer, Stage 2 pancreatic cancer, or Stage 3 pancreatic cancer. In embodiments, the pancreatic cancer is Stage 1 pancreatic cancer, Stage 2 pancreatic cancer, Stage 3 pancreatic cancer, or Stage 4 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent. In embodiments, the method comprises administering to the patient an effective amount of an anti-cancer agent and administering to the patient an effective amount of radiation therapy. In embodiments, the method comprises administering to the patient an effective amount of an anti-cancer agent and surgically removing all or a portion of the pancreas of the patient, or a combination of two or more thereof. In embodiments, the method comprises administering to the patient an effective amount of radiation therapy. In embodiments, the method comprises surgically removing all or a portion of the pancreas of the patient. In embodiments, the method comprises administering to the patient an effective amount of an anti-cancer agent, administering to the patient an effective amount of radiation therapy, and surgically removing all or a portion of the pancreas of the patient. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the method further comprises detecting an elevated expression level, relative to a control, of an RNA selected from the group consisting of cell-free let-7e-5p, cell-free miR-26a-5p, cell-free miR-223-3p, cell-free miR-340-3p, cell-free let-7f-5p, cell-free miR-369-3p, cell-free miR-125a-5p, cell-free miR-495-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-375-3p, exosomal miR-199a-5p, and a combination of two or more thereof. In embodiments, the method further comprises detecting an elevated expression level, relative to a control, of an RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, and a combination of two or more thereof. In embodiments, the method further comprises detecting an elevated expression level, relative to a control, of an RNA selected from the group consisting of cell-free let-7f-5p, cell-free miR-369-3p, cell-free miR-125a-5p, cell-free miR-495-3p, exosomal miR-375-3p, exosomal miR-199a-5p, and a combination of two or more thereof.

Provided herein are methods of detecting an RNA biomarker in a patient having, or suspected of having, pancreatic cancer comprising detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, or a combination of two or more thereof; and wherein the RNA is the RNA biomarker. In embodiments, the patient has pancreatic cancer. In embodiments, the patient is suspected of having pancreatic cancer. The elevated expression level of the RNA, relative to a control, indicates that the patient has pancreatic cancer. In embodiments, The elevated expression level of the RNA, relative to a control, indicates that the patient is likely to have pancreatic cancer. In embodiments, the method further comprises administering to the patient an effective amount of an anti-cancer agent, administering to the patient an effective amount of radiation therapy, surgically removing all or a portion of the pancreas of the patient, or a combination of two or more thereof. In embodiments, the RNA comprises at least two RNA selected from the group consisting of cell-free miR-30c-5p, cell-free miR-142-3p, cell-free miR-340-5p, cell-free miR-335-5p, and cell-free miR-23b-3p. In embodiments, the RNA comprises at least three RNA selected from the group consisting of cell-free miR-30c-5p, cell-free miR-142-3p, cell-free miR-340-5p, cell-free miR-335-5p, and cell-free miR-23b-3p. In embodiments, the RNA comprises at least four RNA selected from the group consisting of cell-free miR-30c-5p, cell-free miR-142-3p, cell-free miR-340-5p, cell-free miR-335-5p, and cell-free miR-23b-3p. In embodiments, the RNA comprises cell-free miR-30c-5p, cell-free miR-142-3p, cell-free miR-340-5p, cell-free miR-335-5p, and cell-free miR-23b-3p. In embodiments, the RNA comprises two RNA selected from the group consisting of cell-free miR-30c-5p, cell-free miR-142-3p, cell-free miR-340-5p, cell-free miR-335-5p, and cell-free miR-23b-3p. In embodiments, the RNA comprises three RNA selected from the group consisting of cell-free miR-30c-5p, cell-free miR-142-3p, cell-free miR-340-5p, cell-free miR-335-5p, and cell-free miR-23b-3p. In embodiments, the RNA comprises four RNA selected from the group consisting of cell-free miR-30c-5p, cell-free miR-142-3p, cell-free miR-340-5p, cell-free miR-335-5p, and cell-free miR-23b-3p. In embodiments, the RNA comprises cell-free miR-30c-5p, cell-free miR-142-3p, cell-free miR-340-5p, cell-free miR-335-5p, and cell-free miR-23b-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the pancreatic cancer is Stage 3 pancreatic cancer or Stage 4 pancreatic cancer. In embodiments, the pancreatic cancer is Stage 3 pancreatic cancer. In embodiments, the pancreatic cancer is Stage 4 pancreatic cancer. In embodiments, the pancreatic cancer is Stage 1 pancreatic cancer, Stage 2 pancreatic cancer, or Stage 3 pancreatic cancer. In embodiments, the pancreatic cancer is Stage 1 pancreatic cancer, Stage 2 pancreatic cancer, Stage 3 pancreatic cancer, or Stage 4 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method further comprises administering to the patient an effective amount of an anti-cancer agent. In embodiments, the method further comprises administering to the patient an effective amount of an anti-cancer agent and administering to the patient an effective amount of radiation therapy. In embodiments, the method further comprises administering to the patient an effective amount of an anti-cancer agent and surgically removing all or a portion of the pancreas of the patient, or a combination of two or more thereof. In embodiments, the method further comprises administering to the patient an effective amount of radiation therapy. In embodiments, the method further comprises surgically removing all or a portion of the pancreas of the patient, or a combination of two or more thereof. In embodiments, the method further comprises administering to the patient an effective amount of an anti-cancer agent, administering to the patient an effective amount of radiation therapy, and surgically removing all or a portion of the pancreas of the patient. In embodiments, the method further comprises detecting an elevated expression level, relative to a control, of an RNA selected from the group consisting of cell-free let-7e-5p, cell-free miR-26a-5p, cell-free miR-223-3p, cell-free miR-340-3p, cell-free let-7f-5p, cell-free miR-369-3p, cell-free miR-125a-5p, cell-free miR-495-3p, and a combination of two or more thereof. In embodiments, the method further comprises detecting an elevated expression level, relative to a control, of an RNA selected from the group consisting of cell-free let-7e-5p, cell-free miR-26a-5p, cell-free miR-223-3p, cell-free miR-340-3p, and a combination of two or more thereof. In embodiments, the method further comprises detecting an elevated expression level, relative to a control, of an RNA selected from the group consisting of cell-free let-7f-5p, cell-free miR-369-3p, cell-free miR-125a-5p, cell-free miR-495-3p, and a combination of two or more thereof.

Provided herein are methods of detecting an RNA biomarker in a patient having, or suspected of having, pancreatic cancer comprising detecting an elevated expression level, relative to a control, of an RNA in a biological sample obtained from the patient, wherein the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof; and wherein the RNA is the RNA biomarker. In embodiments, the patient has pancreatic cancer. In embodiments, the patient is suspected of having pancreatic cancer. The elevated expression level of the RNA, relative to a control, indicates that the patient has pancreatic cancer. In embodiments, The elevated expression level of the RNA, relative to a control, indicates that the patient is likely to have pancreatic cancer. In embodiments, the method further comprises administering to the patient an effective amount of an anti-cancer agent, administering to the patient an effective amount of radiation therapy, surgically removing all or a portion of the pancreas of the patient, or a combination of two or more thereof. In embodiments, the RNA comprises at least two RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage LIB. In embodiments, the pancreatic cancer is Stage 3 pancreatic cancer or Stage 4 pancreatic cancer. In embodiments, the pancreatic cancer is Stage 3 pancreatic cancer. In embodiments, the pancreatic cancer is Stage 4 pancreatic cancer. In embodiments, the pancreatic cancer is Stage 1 pancreatic cancer, Stage 2 pancreatic cancer, or Stage 3 pancreatic cancer. In embodiments, the pancreatic cancer is Stage 1 pancreatic cancer, Stage 2 pancreatic cancer, Stage 3 pancreatic cancer, or Stage 4 pancreatic cancer. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method further comprises administering to the patient an effective amount of an anti-cancer agent. In embodiments, the method further comprises administering to the patient an effective amount of an anti-cancer agent and administering to the patient an effective amount of radiation therapy. In embodiments, the method further comprises administering to the patient an effective amount of an anti-cancer agent and surgically removing all or a portion of the pancreas of the patient, or a combination of two or more thereof. In embodiments, the method further comprises administering to the patient an effective amount of radiation therapy. In embodiments, the method comprises surgically removing all or a portion of the pancreas of the patient. In embodiments, the method further comprises administering to the patient an effective amount of an anti-cancer agent, administering to the patient an effective amount of radiation therapy, and surgically removing all or a portion of the pancreas of the patient. In embodiments, the method comprises administering to the patient image-based screening, administering to the patient an effective amount of an anti-cancer agent, and administering to the patient an effective amount of radiation therapy. In embodiments, the method comprises administering to the patient image-based screening and administering to the patient an effective amount of an anti-cancer agent. In embodiments, the method comprises administering to the patient image-based screening. In embodiments, the method further comprises detecting an elevated expression level, relative to a control, of an RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-375-3p, exosomal miR-199a-5p, and a combination of two or more thereof. In embodiments, the method further comprises detecting an elevated expression level, relative to a control, of an RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, and a combination of two or more thereof. In embodiments, the method further comprises detecting an elevated expression level, relative to a control, of an RNA selected from the group consisting of exosomal miR-375-3p, exosomal miR-199a-5p, and a combination of two or more thereof.

Provided herein are methods of detecting an RNA biomarker in a patient having, or suspected of having, pancreatic cancer comprising detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof, and wherein the RNA is the RNA biomarker. In embodiments, the elevated expression level of the RNA biomarker indicates that the patient has pancreatic cancer. In embodiments, the methods further comprise administering to the patient an effective amount of an anti-cancer agent, administering to the patient an effective amount of radiation therapy, surgically removing all or a portion of the pancreas of the patient, or a combination of two or more thereof. In embodiments, the methods further comprise administering to the patient an effective amount of an anti-cancer agent. In embodiments, the pancreatic cancer is pancreatic ductal adenocarcinoma. In embodiments, the pancreatic cancer is Stage 1 pancreatic cancer or Stage 2 pancreatic cancer. In embodiments, the biological sample obtained from the patient has a normal level of CA19-9. In embodiments, the RNA comprises cell-free miR-30c-5p, cell-free miR-142-3p, cell-free miR-340-5p, cell-free miR-335-5p, cell-free miR-23b-3p, cell-free let-7e-5p, cell-free miR-26a-5p, cell-free miR-223-3p, cell-free miR-340-3p, cell-free let-7f-5p, cell-free miR-369-3p, cell-free miR-125a-5p, cell-free miR-495-3p, or a combination of two or more thereof. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-375-3p, exosomal miR-199a-5p, or a combination of two or more thereof. In embodiments, the control is the average expression level of RNA in a population of healthy patients.

Provided herein are methods of detecting an RNA biomarker in a patient having, or suspected of having, pancreatic cancer comprising detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof; and wherein the RNA is the RNA biomarker. In embodiments, the RNA comprises at least two RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eight RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least nine RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least ten RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least eleven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least twelve RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least thirteen RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eight RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises nine RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises ten RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises eleven RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises twelve RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises thirteen RNA selected from the group consisting of cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage lB. In embodiments, the pancreatic cancer is Stage 2 pancreatic cancer. In embodiments, the pancreatic cancer is Stage IIA. In embodiments, the pancreatic cancer is Stage IIB. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent, surgically removing all or a portion of the pancreas of the patient, or a combination thereof.

Provided herein are methods of detecting an RNA biomarker in a patient having, or suspected of having, pancreatic cancer comprising detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, cell-free let-7e-5p, cell-free miR-26a-5p, cell-free miR-223-3p, cell-free miR-340-3p, or a combination of two or more thereof; and wherein the RNA is the RNA biomarker. In embodiments, the RNA comprises at least two RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises at least three RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises at least four RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises at least five RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises two RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises three RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises four RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises five RNA selected from the group consisting of cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the RNA comprises cell-free let-7e-5p, cell-free miR-26a-5p, cell-free miR-223-3p, cell-free miR-340-3p, cell-free miR-30c-5p, cell-free miR-142-3p, cell-free miR-340-5p, cell-free miR-335-5p, and cell-free miR-23b-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage IIB. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent, surgically removing all or a portion of the pancreas of the patient, or a combination thereof.

Provided herein are methods of detecting an RNA biomarker in a patient having, or suspected of having, pancreatic cancer comprising detecting an elevated expression level, relative to a control, of an RNA in a biological sample obtained from the patient, wherein the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof; and wherein the RNA is the RNA biomarker. In embodiments, the RNA comprises at least two RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least three RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least four RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least five RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least six RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises at least seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises two RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises three RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises four RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises five RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises six RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises seven RNA selected from the group consisting of exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the RNA comprises exosomal miR-1260a, exosomal miR-141-3p, exosomal miR-143-3p, exosomal miR-148a-3p, exosomal miR-200c-3p, exosomal miR-216a-5p, exosomal miR-34a-5p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p. In embodiments, the patient has a symptom of pancreatic cancer. In embodiments, the patient has new-onset diabetes. In embodiments, the patient has diabetes. In embodiments, the patient is at least 45 years old. In embodiments, the patient has new-onset diabetes and is at least 50 years old. In embodiments, the human patient is at least 55 years old. In embodiments, the human patient is at least 60 years old. In embodiments, the patient has diabetes (e.g., new-onset diabetes or pre-existing diabetes), has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof. In embodiments, the patient has obesity. In embodiments, the patient smokes nicotine (e.g., cigarettes, cigars, e-cigarettes) or has a history of smoking nicotine. In embodiments, the patient has a history of pancreatitis. In embodiments, the patient has a history of chronic pancreatitis. In embodiments, the pancreatic cancer is pancreatic ductal adenocarcinoma. In embodiments, the pancreatic cancer is Stage 1 pancreatic cancer or Stage 2 pancreatic cancer. In embodiments, the pancreatic cancer is Stage 1 pancreatic cancer. In embodiments, the pancreatic cancer is Stage IA. In embodiments, the pancreatic cancer is Stage IB. In embodiments, the pancreatic cancer is Stage 2 pancreatic cancer. In embodiments, the pancreatic cancer is Stage IIA. In embodiments, the pancreatic cancer is Stage LIB. In embodiments, the biological sample obtained from the patient has a normal level of CA19-9. In embodiments, the biological sample obtained from the patient has an elevated level of CA19-9. In embodiments, the biological sample obtained from the patient is Lewis antigen-negative. In embodiments, the biological sample is a blood sample. In embodiments, the blood sample is a serum sample. In embodiments, the blood sample is a plasma sample. In embodiments, the control is the average expression level of RNA in a population of healthy patients. In embodiments, the method comprises administering to the patient an effective amount of an anti-cancer agent, surgically removing all or a portion of the pancreas of the patient, or a combination thereof.

### Anti-Cancer Agents

In embodiments, the methods described herein comprise administering to a patient an effective amount of an anti-cancer agent. The anticancer treatment can be any drug known in the art as useful for treating cancer, such as chemotherapy, immunotherapy, or a combination thereof. In embodiments, the anti-cancer agent is a chemotherapeutic agent. In embodiments, the anti-cancer agent comprises everolimus, erlotinib, olaparib, mitomycin, sunitinib, gemcitabine, 5-fluorouracil, irinotecan, oxaliplatin, paclitaxel, capecitabine, cisplatin, docetaxel, or a combination of two or more thereof.

In embodiments, the chemotherapeutic agent is an alkylating agent, an antimetabolite compound, an anthracycline compound, an antitumor antibiotic, a platinum compound, a topoisomerase inhibitor, a vinca alkaloid, a taxane compound, an epothilone compound, or a combination of two or more thereof. In embodiments, the alkylating agent is carboplatin, chlorambucil, cyclophosphamide, melphalan, mechlorethamine, procarbazine, or thiotepa. In embodiments, the antimetabolite compound is azacitidine, capecitabine, cytarabine, gemcitabine, doxifluridine, hydroxyurea, methotrexate, pemetrexed, 6-thioguanine, 5-fluorouracil, or 6-mercaptopurine. In embodiments, the anthracycline compound is daunorubicin, doxorubicin, idarubicin, epirubicin, or mitoxantrone. In embodiments, the antitumor antibiotic is actinomycin, bleomycin, mitomycin, or valrubicin. In embodiments, the platinum compound is cisplatin or oxaliplatin. In embodiments, the topoisomerase inhibitor is irinotecan, topotecan, amsacrine, etoposide, teniposide, or eribulin. In embodiments, the vinca alkaloid is vincristine, vinblastine, vinorelbine, or vindesine. In embodiments, the taxane compound is paclitaxel or docetaxel. In embodiments, the epothilone compound is epothilone, ixabepilone, patupilone, or sagopilone.

In embodiments, the method comprises administering to the subject an effective amount of gemcitabine, 5-fluorouracil, irinotecan, oxaliplatin, paclitaxel, capecitabine, cisplatin, docetaxel, or a combination of two or more thereof. In embodiments, the method comprises administering to the subject an effective amount of gemcitabine. In embodiments, the method comprises administering to the subject an effective amount of 5-fluorouracil. In embodiments, the method comprises administering to the subject an effective amount of irinotecan. In embodiments, the method comprises administering to the subject an effective amount of oxaliplatin. In embodiments, the method comprises administering to the subject an effective amount of paclitaxel. In embodiments, the method comprises administering to the subject an effective amount of capecitabine. In embodiments, the method comprises administering to the subject an effective amount of cisplatin. In embodiments, the method comprises administering to the subject an effective amount of docetaxel. In embodiments, the method comprises or further comprises administering to the subject an effective amount of leucovorin.

“Chemotherapeutic” or “chemotherapeutic agent” is used in accordance with its plain ordinary meaning and refers to a chemical composition or compound having antineoplastic properties or the ability to inhibit the growth or proliferation of cells.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, C1-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec®), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein kinase C inhibitors, protein tyrosine phosphatase inhibitors; purine nucleoside phosphorylase inhibitors; ras famesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

Additionally, the compounds described herein can be co-administered with conventional immunotherapeutic agents including, but not limited to, immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y, or ¹³¹I, etc.).

### Kits

Provided here are kits comprising components, such as reagents and reaction mixtures, to conduct the assays to detect the miRNA and mRNA as described herein. As part of the kit, materials and instruction are provided, e.g., for storage and use of kit components. In embodiments, the kits comprise one or more of the following: a RNA probe that can hybridize to a RNA biomarker, pairs of primers that under appropriate reaction conditions can prime amplification of at least a portion of a RNA marker or a RNA encoding a polypeptide marker (e.g., by PCR), instructions on how to use the kit, and a label or insert indicating regulatory approval for diagnostic or therapeutic use. In embodiments, the kit further includes RNA microarrays comprising RNA of the disclosure or molecules which specifically bind to the RNA described herein. In embodiments, standard techniques of microarray technology are utilized to assess expression of the RNA. Polynucleotide arrays, particularly arrays that bind RNA described herein, also can be used for diagnostic applications, such as for identifying subjects that have a condition characterized by expression of polypeptide biomarkers.

“Assaying” or “detecting” means using an analytic procedure to qualitatively assess or quantitatively measure the presence or amount or the functional activity of a target entity (e.g., miRNA, mRNA). For example, detecting the level of RNA (such as miRNA or mRNA) means using an analytic procedure (such as an in vitro procedure) to qualitatively assess or quantitatively measure the presence or amount of the RNA. In embodiments, raw expression values are normalized by performing quantile normalization relative to the reference distribution and subsequent log 10-transformation. In embodiments, when RNA expression is detected using the nCounter® Analysis System marketed by Nanostring Technologies, the reference distribution is generated by pooling reported (i.e., raw) counts for the test sample and one or more control samples (preferably at least 2 samples, more preferably at least any of 4, 8 or 16 samples) after excluding values for technical (both positive and negative control) probes and without performing intermediate normalization relying on negative (background-adjusted) or positive (synthetic sequences spiked with known titrations).

The terms “probe” or “primer” refer to one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe or primers can be unlabeled or labeled as described below so that its binding to a target sequence can be detected (e.g., with a FRET donor or acceptor label). The probe or primer can be designed based on one or more particular (preselected) portions of a chromosome, e.g., one or more clones, an isolated whole chromosome or chromosome fragment, or a collection of polymerase chain reaction (PCR) amplification products. One of skill can adjust these factors to provide optimum hybridization and signal production for a given hybridization and detection procedures, and to provide the required resolution among different genes or genomic locations.

Probes and primers can also be immobilized on a solid surface (e.g., nitrocellulose, glass, quartz, fused silica slides), as in an array. Techniques for producing high density arrays can also be used for this purpose. One of skill will recognize that the precise sequence of particular probes and primers can be modified from the target sequence to a certain degree to produce probes that are “substantially identical” or “substantially complementary to” a target sequence, but retain the ability to specifically bind to (i.e., hybridize specifically to) the same targets from which they were derived.

The term “capable of hybridizing to” refers to a polynucleotide sequence that forms Watson-Crick bonds with a complementary sequence. One of skill will understand that the percent complementarity need not be 100% for hybridization to occur, depending on the length of the polynucleotides, length of the complementary region (e.g. 5, 10, 15, 20, 25, 30, 35, 40, 45, 50, 60, 70, 80, 90, 100, or more bases in length), and stringency of the conditions. For example, a polynucleotide (e.g., primer or probe) can be capable of binding to a polynucleotide having 60%, 65%, 70%, 75%, 80%, 85%, 90%, 91%, 92%, 93%, 94%, 95%, 96%, 97%, 98%, 99% or 100% complementarity over the stretch of the complementary region.

In embodiments, methods include detecting a level of a biomarker with a specific binding agent (e.g., an agent that binds to a protein or nucleic acid molecule). Exemplary binding agents include an antibody or a fragment thereof, a detectable protein or a fragment thereof, a nucleic acid molecule such as an oligonucleotide/polynucleotide comprising a sequence that is complementary to patient genomic DNA, mRNA or a cDNA produced from patient mRNA, or any combination thereof. In embodiments, an antibody is labeled with detectable moiety, e.g., a fluorescent compound, an enzyme or functional fragment thereof, or a radioactive agent. In embodiments, an antibody is detectably labeled by coupling it to a chemiluminescent compound. In embodiments, the presence of the chemiluminescent-tagged antibody is then determined by detecting the presence of luminescence that arises during the course of chemical reaction. Non-limiting examples of useful chemiluminescent labeling compounds are luminol, isoluminol, theromatic acridinium ester, imidazole, acridinium salt and oxalate ester.

In embodiments, a specific binding agent is an agent that has greater than 10-fold, preferably greater than 100-fold, and most preferably, greater than 1000-fold affinity for the target molecule as compared to another molecule. As the skilled artisan will appreciate the term specific is used to indicate that other biomarkers present in the sample do not significantly bind to the binding agent specific for the target molecule. In embodiments, the level of binding to a biomolecule other than the target biomarker results in a binding affinity which is at most only 10% or less, only 5% or less only 2% or less or only 1% or less of the affinity to the target molecule, respectively. A preferred specific binding agent will fulfill both the above minimum criteria for affinity as well as for specificity. For example, in embodiments an antibody has a binding affinity (e.g., Kd) in the low micromolar (10⁻⁶), nanomolar (10⁻⁷-10⁻⁹), with high affinity antibodies in the low nanomolar (10⁻¹) or picomolar (10⁻¹²) range for its specific target biomarker.

In embodiments, the subject matter provides a composition comprising a binding agent, wherein the binding agent is attached to a solid support, (e.g., a strip, a polymer, a bead, a nanoparticle, a plate such as a multiwell plate, or an array such as a microarray). In embodiments relating to the use of a nucleic acid probe attached to a solid support (such as a microarray), a nucleic acid in a test sample may be amplified (e.g., using PCR) before or after the nucleic acid to be measured is hybridized with the probe. In embodiments, reverse transcription polymerase chain reaction (RT-PCR) is used to detect mRNA levels. In embodiments, a probe on a solid support is used, and mRNA (or a portion thereof) in a biological sample is converted to cDNA or partial cDNA and then the cDNA or partial cDNA is hybridized to a probe (e.g., on a microarray), hybridized to a probe and then amplified, or amplified and then hybridized to a probe. In embodiments, a strip may be a nucleic acid-probe coated porous or non-porous solid support strip comprising linking a nucleic acid probe to a carrier to prepare a conjugate and immobilizing the conjugate on a porous solid support. In embodiments, the support or carrier comprises glass, polystyrene, polypropylene, polyethylene, dextran, nylon, amylases, natural and modified celluloses, polyacrylamides, gabbros, and magnetite. In embodiments, the nature of the carrier can be either soluble to some extent or insoluble for the purposes of the present subject matter. In embodiments, the support material may have any structural configuration so long as the coupled molecule is capable of binding to a binding agent (e.g., an antibody). In embodiments, the support configuration may be spherical, as in a bead, or cylindrical, as in the inside surface of a test tube, or the external surface of a rod. In embodiments, the surface may be flat such as a plate (or a well within a multiwell plate), sheet, test strip, polystyrene beads. Those skilled in the art will know many other suitable carriers for binding antibody or antigen, or will be able to ascertain the same by use of routine experimentation.

In embodiments, a solid support comprises a polymer, to which an agent is chemically bound, immobilized, dispersed, or associated. In embodiments, a polymer support may be, e.g., a network of polymers, and may be prepared in bead form (e.g., by suspension polymerization). In embodiments, the location of active sites introduced into a polymer support depends on the type of polymer support. In embodiments, in a swollen-gel-bead polymer support the active sites are distributed uniformly throughout the beads, whereas in a macroporous-bead polymer support they are predominantly on the internal surfaces of the macropores. In embodiments, the solid support, e.g., a device, may contain a biomarker binding agent alone or together with a binding agent for at least one, two, three or more other biomarkers.

In embodiments, the cells in a biological sample are lysed to release a protein or nucleic acid. Numerous methods for lysing cells and assessing protein and nucleic acid levels are known in the art. In embodiments, cells are physically lysed, such as by mechanical disruption, liquid homogenization, high frequency sound waves, freeze/thaw cycles, with a detergent, or manual grinding. Non-limiting examples of detergents include Tween 20, Triton X-100, and sodium dodecyl sulfate (SDS). Non-limiting examples of assays for determining the level of a protein include HPLC, LC/MS, ELISA, immunoelectrophoresis, Western blot, immunohistochemistry, and radioimmunoassays. Non-limiting examples of assays for determining the level of an mRNA include Northern blotting, RT-PCR, RNA sequencing, and qRT-PCR.

In embodiments, once a suitable biological sample has been obtained, it is analyzed to quantitate the expression level of each of the biomarker genes. In embodiments, determining the expression level of a gene comprises detecting and quantifying RNA transcribed from that gene or a protein translated from such RNA. In embodiments, the RNA includes mRNA transcribed from the gene, and/or specific spliced variants thereof and/or fragments of such mRNA and spliced variants.

In embodiments, raw expression values are normalized by performing quantile normalization relative to the reference distribution and subsequent log 10-transformation. In embodiments, when the gene expression is detected using the nCounter@Analysis System marketed by NanoString@Technologies, the reference distribution is generated by pooling reported (i.e., raw) counts for the test sample and one or more control samples (preferably at least 2 samples, more preferably at least any of 4, 8 or 16 samples) after excluding values for technical (both positive and negative control) probes and without performing intermediate normalization relying on negative (background-adjusted) or positive (synthetic sequences spiked with known titrations). In embodiments, the T-effector signature score is then calculated as the arithmetic mean of normalized values for each of the genes in the gene signature.

A “detectable agent” or “detectable moiety” is a compound or composition detectable by appropriate means such as spectroscopic, photochemical, biochemical, immunochemical, chemical, magnetic resonance imaging, or other physical means. The RNA described herein and the expression level of the RNA described herein may be accomplished through the use of a detectable moiety in an assay or kit. A detectable moiety is a monovalent detectable agent or a detectable agent bound (e.g. covalently and directly or via a linking group) with another compound, e.g., a nucleic acid. Exemplary detectable agents/moieties for use in the present disclosure include an antibody ligand, a peptide, a nucleic acid, radioisotopes, paramagnetic metal ions, fluorophore (e.g. fluorescent dyes), electron-dense reagents, enzymes (e.g., as commonly used in an ELISA), biotin, a biotin-avidin complex, a biotin-streptavidin complex, digoxigenin, magnetic beads (e.g., DYNABEADS® by ThermoFisher, encompassing functionalized magnetic beads such as DYNABEADS® M-270 amine by ThermoFisher), paramagnetic molecules, paramagnetic nanoparticles, ultrasmall superparamagnetic iron oxide nanoparticles, ultrasmall superparamagnetic iron oxide nanoparticle aggregates, superparamagnetic iron oxide nanoparticles, superparamagnetic iron oxide nanoparticle aggregates, monocrystalline iron oxide nanoparticles, monocrystalline iron oxide, nanoparticle contrast agents, liposomes or other delivery vehicles containing Gadolinium chelate molecules, gadolinium, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

In embodiments, oligonucleotides in kits are capable of specifically hybridizing to a target region of a polynucleotide, such as for example, an RNA transcript or cDNA generated therefrom. As used herein, specific hybridization means the oligonucleotide forms an anti-parallel double-stranded structure with the target region under certain hybridizing conditions, while failing to form such a structure with non-target regions when incubated with the polynucleotide under the same hybridizing conditions. The composition and length of each oligonucleotide in the kit will depend on the nature of the transcript containing the target region as well as the type of assay to be performed with the oligonucleotide and is readily determined by the skilled artisan.

In embodiments, the kit comprises reagents capable of detecting an expression level of RNA from a blood sample; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof. In embodiments, the RNA comprises cell-free miR-30c-5p, cell-free miR-142-3p, cell-free miR-340-5p, cell-free miR-335-5p, and cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p.

In embodiments, the kit comprises reagents capable of detecting an expression level of RNA from a blood sample; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof. In embodiments, the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, and exosomal miR-34a-5p.

In embodiments, the kit comprises reagents capable of detecting an expression level of RNA from a blood sample; wherein the RNA comprises cell-free miR-30c-5p, cell-free miR-142-3p, cell-free miR-340-5p, cell-free miR-335-5p, cell-free miR-23b-3p, or a combination of two or more thereof. In embodiments, the RNA comprises cell-free miR-30c-5p, cell-free miR-142-3p, cell-free miR-340-5p, cell-free miR-335-5p, and cell-free miR-23b-3p.

In embodiments, the kit comprises reagents capable of detecting an expression level of RNA from a blood sample; wherein the RNA comprises cell-free miR-30c-5p, cell-free miR-142-3p, cell-free miR-340-5p, cell-free miR-335-5p, cell-free miR-23b-3p, cell-free let-7e-5p, cell-free miR-26a-5p, cell-free miR-223-3p, cell-free miR-340-3p, or a combination of two or more thereof. In embodiments, the RNA comprises cell-free miR-30c-5p, cell-free miR-142-3p, cell-free miR-340-5p, cell-free miR-335-5p, cell-free miR-23b-3p, cell-free let-7e-5p, cell-free miR-26a-5p, cell-free miR-223-3p, and cell-free miR-340-3p.

In embodiments, the kit comprises reagents capable of detecting an expression level of RNA from a blood sample; wherein the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p.

In embodiments, the kit comprises reagents capable of detecting an expression level of RNA from a blood sample; wherein the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof. In embodiments, the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, exosomal miR-1260a, exosomal miR-141-3p, exosomal miR-143-3p, exosomal miR-148a-3p, exosomal miR-200c-3p, exosomal miR-216a-5p, and exosomal miR-34a-5p.

In embodiments, the disclosure provides a kit for detecting the RNA (e.g., miRNA, mRNA) described herein. In embodiments, the kit is an assay system including any one of assay reagents, assay controls, protocols, exemplary assay results, or combinations of these components designed to provide the user with means to evaluate the expression level of the RNA (e.g., miRNA, mRNA) described herein.

In embodiments, the disclosure provides a kit for diagnosing pancreatic cancer in a patent, including reagents for detecting RNA markers in a biological (e.g., blood) sample from a patient.

In embodiments, the kits comprise one or more of the following: a RNA probe that can hybridize to a RNA biomarker, pairs of primers that under appropriate reaction conditions can prime amplification of at least a portion of a RNA marker or a RNA encoding a polypeptide marker (e.g., by PCR), instructions on how to use the kit, and a label or insert indicating regulatory approval for diagnostic or therapeutic use.

In embodiments, the kit further includes RNA microarrays comprising RNA of the disclosure or molecules which specifically bind to the RNA described herein. In embodiments, standard techniques of microarray technology are utilized to assess expression of the RNA. Polynucleotide arrays, particularly arrays that bind RNA described herein, also can be used for diagnostic applications, such as for identifying subjects that have a condition characterized by expression of polypeptide biomarkers, e.g., interstitial lung disease.

In addition, the means for detecting of the assay system of the present disclosure can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide). The kit can also include suitable reagents for the detection of the reagent and/or for the labeling of positive or negative controls, wash solutions, dilution buffers and the like. The assay system can also include a set of written instructions for using the system and interpreting the results.

### Gene Expression

In embodiments of the methods described herein, the elevated level of gene expression is an elevated level of RNA (e.g., miRNA) expression. Levels of gene expression can be determined by methods known in the art, such as those described herein. In embodiments, the RNA is miRNA. In embodiments, RNA expression is detected by direct digital counting of nucleic acids, RNA sequencing (RNA-seq), quantitative reverse transcriptase polymerase chain reaction (RT-qPCR), quantitative polymerase chain reaction (qPCR), multiplex qPCR, microarray analysis, or a combination thereof. In embodiments, RNA expression is detected by RNA sequencing. RNA sequencing is a sequencing technique which uses next-generation sequencing (NGS) to reveal the presence and quantity of RNA in a biological sample. In embodiments, the gene expression level is an average of the gene expression level of the biomarker genes. In embodiments, the average of the gene expression level of the biomarker genes is an average of the normalized gene expression level of the biomarker genes. In embodiments, the gene expression level of the biomarker genes is a median of the gene expression level of the biomarker genes. In embodiments, the median of the gene expression level of the biomarker genes is a median of a normalized gene expression level of the biomarker genes. In embodiments, the gene expression level of the biomarker genes is the gene expression level of the biomarker genes normalized to a reference gene.

In embodiments of the methods described herein, the individual elevated expression level of the RNA described herein are used. In embodiments, the individual elevated expression level of the exosomal RNA described herein (e.g., exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p) are used. In embodiments, the individual elevated expression level of the cell-free RNA described herein (e.g., cell-free miR-30c-5p, cell-free miR-142-3p, cell-free miR-340-5p, cell-free miR-335-5p, cell-free miR-23b-3p) are used. In embodiments, the individual elevated expression level of the cell-free RNA and exosomal RNA described herein (e.g., cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p) are used. In embodiments, the elevated expression levels of the RNA are combined to form a risk score. In embodiments, the elevated expression levels of the exosomal RNA described herein (e.g., exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p) are combined to form a risk score. In embodiments, the elevated expression levels of the cell-free RNA described herein (e.g., cell-free miR-30c-5p, cell-free miR-142-3p, cell-free miR-340-5p, cell-free miR-335-5p, cell-free miR-23b-3p) are combined to form a risk score. In embodiments, the elevated expression levels of the cell-free RNA and exosomal RNA described herein (e.g., cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p) are combined to form a risk score.

In embodiments, the elevated expression levels of the RNA are weighted and combined to form a risk score. In embodiments, the elevated expression levels of the exosomal RNA described herein (e.g., exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p) are weighted and combined to form a risk score. In embodiments, the elevated expression levels of the cell-free RNA described herein (e.g., cell-free miR-30c-5p, cell-free miR-142-3p, cell-free miR-340-5p, cell-free miR-335-5p, cell-free miR-23b-3p) are weighted and combined to form a risk score. In embodiments, the elevated expression levels of the cell-free and exosomal RNA described herein (e.g., cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p) are weighted and combined to form a risk score.

Relative quantification relates the PCR signal of the target transcript in a treatment group to that of another sample such as the control (e.g., healthy patients). The₂-ACt method is a convenient way to analyze the relative changes in gene expression from real-time quantitative PCR experiments. The Ct (threshold cycle) method quantification was used for the evaluation of the expression level of each miRNA. The threshold cycle (Ct) is defined as the PCR cycle at which the fluorescent signal of the reporter dye crosses an arbitrarily placed threshold. This method allows to quantify the absolute expression of each miRNAs in each sample analyzed and then to calculate the different expression of each miRNA in sample versus the controls. These expression values of the RNA can be used individually to produce a risk score, can be added together to produce a risk score, or logistic regression analysis can be applied to produce a risk score based on weighted values of the expression levels of the RNA.

In embodiments, the disclosure provides methods of processing RNA expression data generated from the expression levels of the RNA in the biological sample obtained from a patient as described herein, for establishing the presence of a signature indicative of pancreatic cancer (e.g., PDAC), comprising the steps of (i) normalizing and/or scaling numeric values of the RNA expression data (e.g., the exosomal RNA expression data and/or cell-free RNA expression data), (ii) refining the discriminatory power of individual RNA by statistically weighting some of the numeric values associated therewith, and (iii) summating the numeric values obtained from step (ii) to provide a composite expression score. In embodiments, the composite expression score obtained from step (iii) is compared to a control and the comparison allows the sample to be designated as positive or negative for pancreatic cancer. In embodiments, the composite expression score is normalized. In embodiments, the composite expression score is scaled. In embodiments, the composite expression score is weighted. Weighted refers to the relevant value being adjusted to more appropriately reflect its contribution to the profile.

In embodiments, the risk score for the cell-free RNA is determined by the following formula: cf-miRNA panel-[(3.907893 X cf-miR30c-5p)+(−0.13495 X cf-let7e)+(0.979111 X cf-miR340-5p)+(−0.01936 X cf-miR223-3p)+(−0.38275 X cf-miR-26a-5p)+(0.195893 X cf-miR340-3p)+(0.718554 X cf-miR335-5p)+(−1.75009 X cf-miR23b-3p)+(−1.3107 X cf-miR142-3p)+1.501128]. In embodiments, the risk score for the exosomal RNA is determined by the following formula: exo-miRNA panel-[(−0.03638 X exo-miR200c-3p)+(1.087279 X exo-miR148a-3p)+(1.04184 X exo-miR216a-5p)+(−3.1777 X exo-miR145-5p)+(4.112727 X exo-miR200b-3p)+(0.057046 X exo-miR143-3p)+(0.118696 X exo-miR34a-5p)+(−0.92937 X exo-miR429)+(−0.57374 X exo-miR141-3p)+(−4.10024 X exo-miR1260b)+(2.42878 X exo-miR145-3p)+(0.962229 X exo-miR216b-5p)+(1.189049 X exo-miR200a-3p)+(1.691518 X exo-miR1260a)+(1.622576 X exo-miR217-5p)+2.981741]. In embodiments, risk score is a combination of the risk score for the cell-free RNA is added to the risk score for the exosomal RNA. In embodiments, the expression of level of each miRNA is calculated using 2-Act method, the normalized expression values are log₁₀ transformed, and then used in the equations herein.

In embodiments, the risk score for the cell-free RNA is determined by the following formula: cf-miRNA panel-[(3.8758 X cf-miR30c-5p)+(0.9970 X cf-miR340-5p)+(0.8286 X cf-miR335-5p)+(−1.9845 X cf-miR23b-3p)+(−1.4499 X cf-miR142-3p)+1.5448]. In embodiments, the risk score for the exosomal RNA is determined by the following formula: exo-miRNA panel-[(−2.9317 X exo-miR145-5p)+(3.2009 X exo-miR200b-3p)+(−1.2140 X exo-miR429)+(−1.3622 X exo-miR1260b)+(2.9393 X exo-miR145-3p)+(0.9949 X exo-miR216b)+(1.5168 X exo-miR200a-3p)+(1.4536 X exo-miR217-5p)+2.3454]. In embodiments, risk score is a combination of the risk score for the cell-free RNA is added to the risk score for the exosomal RNA. In embodiments, the expression of level of each miRNA is calculated using 2^{-ΔCt} method, the normalized expression values are log₁₀ transformed, and then used in the equations herein.

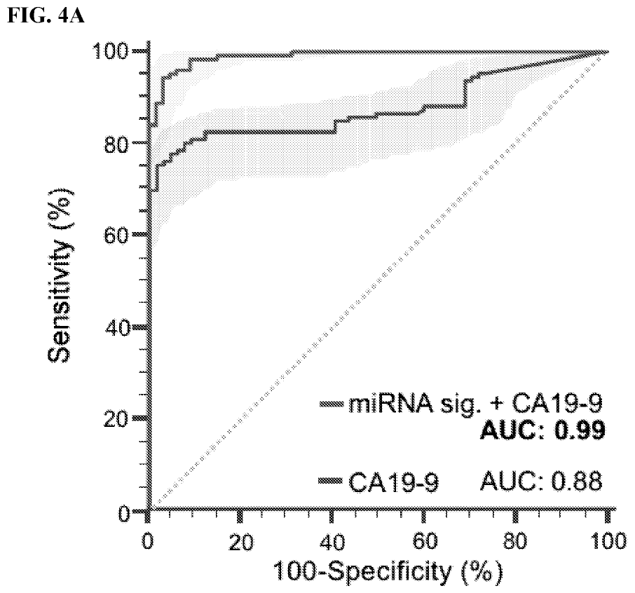

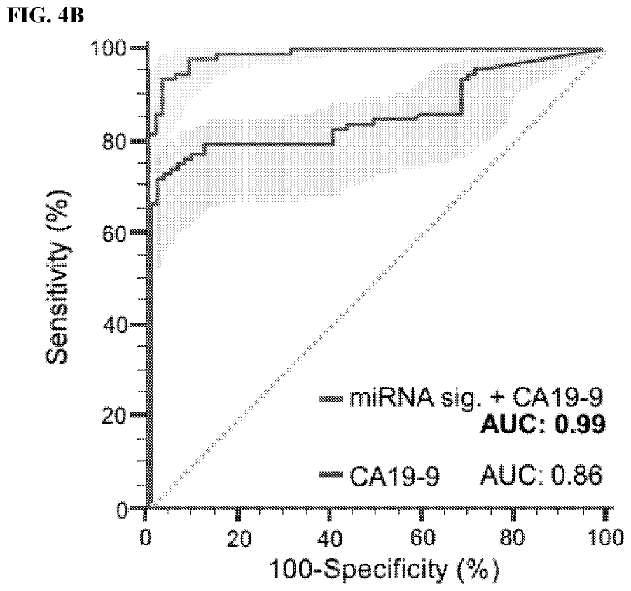

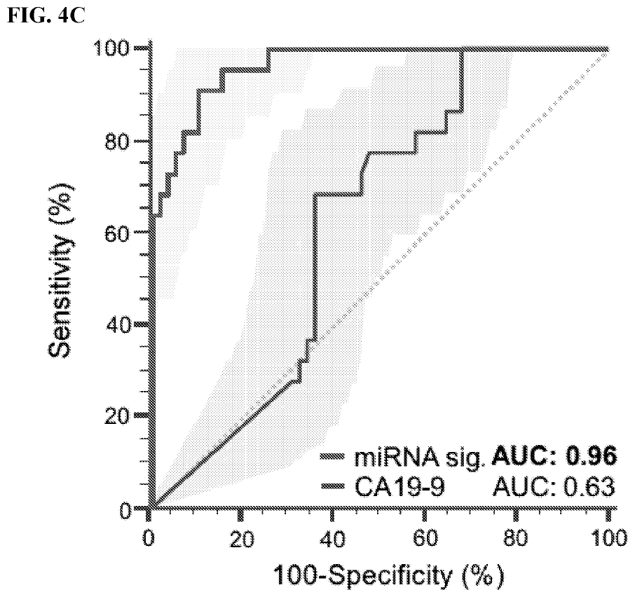

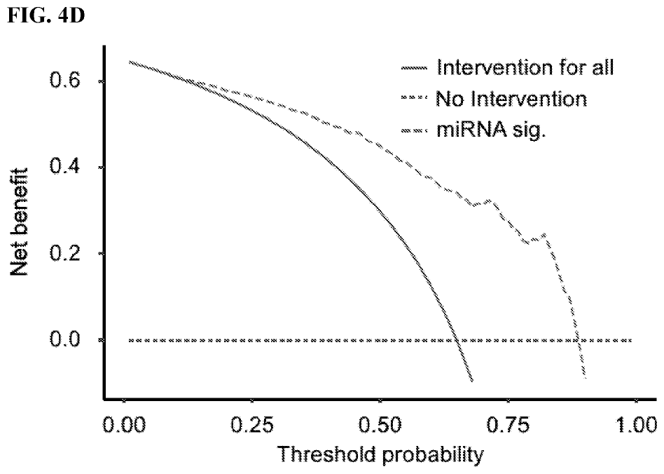

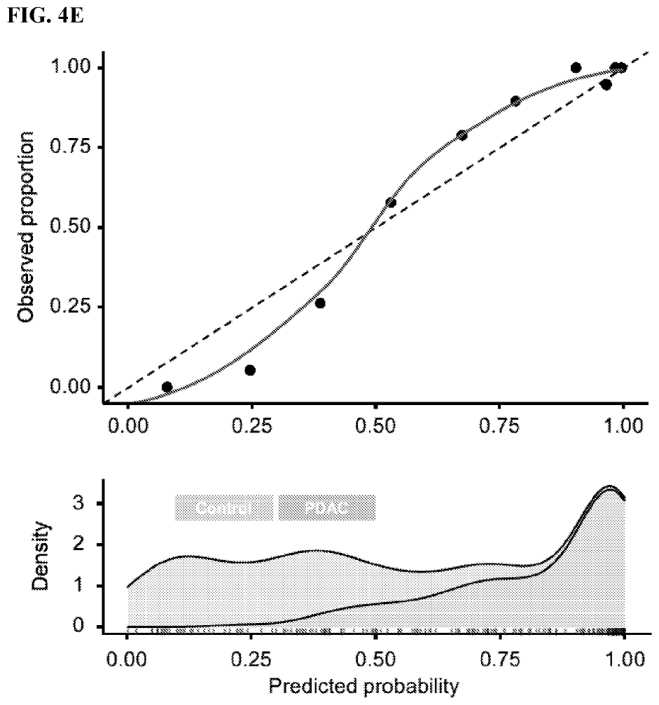

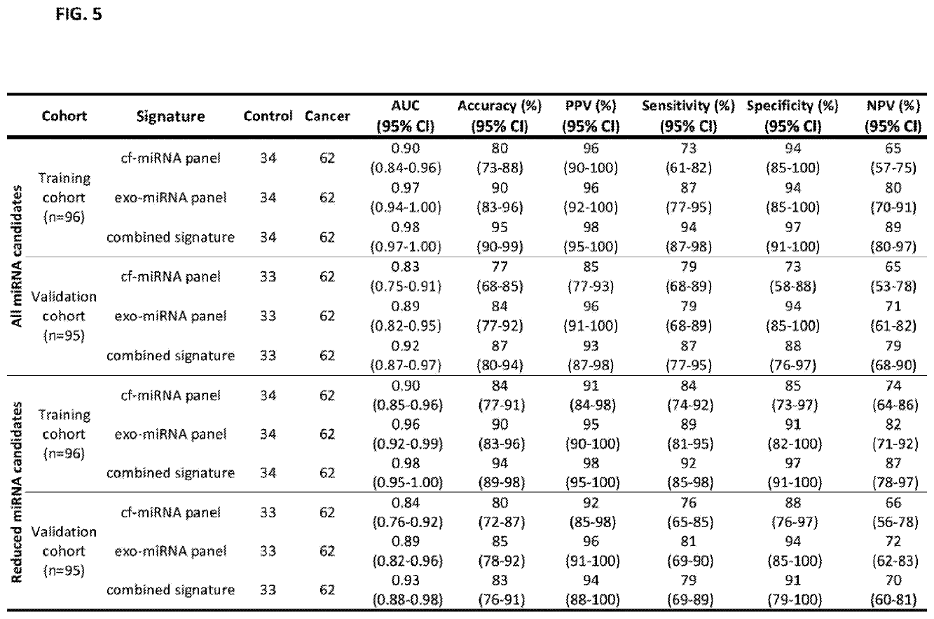

In current clinical practice, diagnosis of PDAC patients are achieved by computed tomography or an invasive biopsy followed by surgery if it is resectable. Accordingly, false positive or false negative cases based on current clinical practice would be detrimental to subjects undergoing this screening. Thus, the clinical usefulness of screening strategies should be estimated by the trade-off between the harm and diagnosis. A decision curve analysis (DCA) and calibration curve analysis revealed that this transcriptomic signature achieved a higher net benefit across most ranges of threshold probability in comparison to diagnosing all PDAC patients or none of the patients. (FIG. 4D). For instance, at threshold probability of 0.50, this transcriptomic signature exhibited a significantly higher net benefit of 0.45 for the diagnosis of PDAC vs. diagnosing all PDAC patients based on the strategy of intervention for all cases with a significantly lower net benefit of approximately 0.30. These findings indicate that the transcriptomic signature offers markedly higher clinical benefit compared to intervention for all cases or none of the cases, in terms of the viewpoint of the avoidance of harm and misdiagnosis. In addition, the calibration plots showed a good agreement between the observed vs. predicted probability across all ranges (FIG. 4E). A slight underestimation for diagnosis of PDAC was observed when the predicted probabilities were between 0.50-0.75 range. As the risk score increases from 0 to 1, the proportion of cancer patients also increases, highlighting that the RNA signature described herein exhibits robust diagnostic identification of patients with pancreatic cancer.

### Embodiments 1-74

Embodiment 1. A method of treating pancreatic cancer in a patient in need thereof, the method comprising administering to the patient an effective amount of an anti-cancer agent, administering to the patient an effective amount of radiation therapy, administering to the patient image-based screening, surgically removing all or a portion of the pancreas of the patient, or a combination of two or more thereof; wherein a biological sample obtained from the patient comprises an elevated expression level, relative to a control, of an RNA; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof.

Embodiment 2. A method of treating pancreatic cancer in a patient in need thereof, the method comprising: (i) detecting an elevated expression level, relative to a control, of an RNA in a biological sample obtained from a patient, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof; and (ii) administering to the patient an effective amount of an anti-cancer agent, administering to the patient an effective amount of radiation therapy, administering to the patient image-based screening, surgically removing all or a portion of the pancreas of the patient, or a combination of two or more thereof.

Embodiment 3. A method of diagnosing a patient with pancreatic cancer, the method comprising detecting an elevated expression level, relative to a control, of an RNA in a biological sample obtained from the patient, thereby diagnosing the patient with pancreatic cancer; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof.

Embodiment 4. A method of monitoring a patient at risk for developing pancreatic cancer, the method comprising: (i) detecting an expression level of an RNA in a biological sample obtained from the patient at a first point in time; (ii) detecting an expression level of an RNA in a biological sample obtained from the patient at a second point in time, wherein the second point in time is later than the first point in time; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof.

Embodiment 5. The method of Embodiment 4, wherein an elevated expression level of RNA at the second point in time when compared to the expression level of RNA at the first point in time indicates that the patient has an increased risk of developing pancreatic cancer.

Embodiment 6. A method of detecting an RNA biomarker in a patient having, or suspected of having, pancreatic cancer, the method comprising detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof.

Embodiment 7. The method of any one of Embodiments 3 to 6, further comprising administering to the patient an effective amount of an anti-cancer agent, administering to the patient an effective amount of radiation therapy, administering to the patient image-based screening, surgically removing all or a portion of the pancreas of the patient, or a combination of two or more thereof.

Embodiment 8. The method of any one of Embodiments 1 to 7, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof.

Embodiment 9. The method of any one of Embodiments 1 to 8, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof.

Embodiment 10. The method of any one of Embodiments 1 to 9, wherein the RNA comprises cell-free miR-30c-5p.

Embodiment 11. The method of any one of Embodiments 1 to 10, wherein the RNA comprises cell-free miR-142-3p.

Embodiment 12. The method of any one of Embodiments 1 to 11, wherein the RNA comprises cell-free miR-340-5p.

Embodiment 13. The method of any one of Embodiments 1 to 12, wherein the RNA comprises cell-free miR-335-5p.

Embodiment 14. The method of any one of Embodiments 1 to 13, wherein the RNA comprises cell-free miR-23b-3p.

Embodiment 15. The method of any one of Embodiments 1 to 14, wherein the RNA comprises exosomal miR-1260b.

Embodiment 16. The method of any one of Embodiments 1 to 15, wherein the RNA comprises exosomal miR-145-5p.

Embodiment 17. The method of any one of Embodiments 1 to 16, wherein the RNA comprises exosomal miR-200a-3p.

Embodiment 18. The method of any one of Embodiments 1 to 17, wherein the RNA comprises exosomal miR-200b-3p.

Embodiment 19. The method of any one of Embodiments 1 to 18, wherein the RNA comprises exosomal miR-216b-5p.

Embodiment 20. The method of any one of Embodiments 1 to 19, wherein the RNA comprises exosomal miR-217-5p.

Embodiment 21. The method of any one of Embodiments 1 to 20, wherein the RNA comprises exosomal miR-429.

Embodiment 22. The method of any one of Embodiments Ito 21, wherein the RNA comprises exosomal miR-145-3p.

Embodiment 23. The method of any one of Embodiments 1 to 9, wherein the RNA comprises cell-free miR-30c-5p, cell-free miR-142-3p, cell-free miR-340-5p, cell-free miR-335-5p, cell-free miR-23b-3p, or a combination of two or more thereof.

Embodiment 24. The method of Embodiment 23, wherein the RNA comprises cell-free miR-30c-5p, cell-free miR-142-3p, cell-free miR-340-5p, cell-free miR-335-5p, and cell-free miR-23b-3p.

Embodiment 25. The method of any one of Embodiments 1 to 9, wherein the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof.

Embodiment 26. The method of Embodiment 25, wherein the RNA comprises exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p.

Embodiment 27. The method of any one of Embodiments 1 to 9, wherein the RNA comprises cell-free miR-30c-5p, cell-free miR-142-3p, cell-free miR-340-5p, cell-free miR-335-5p, and cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p.

Embodiment 28. The method of any one of Embodiments 1 to 9, wherein the RNA consists of cell-free miR-30c-5p, cell-free miR-142-3p, cell-free miR-340-5p, cell-free miR-335-5p, and cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p.

Embodiment 29. The method of any one of Embodiments 1 to 28, further comprising detecting a level of carbohydrate antigen 19-9 (CA19-9) in the biological sample obtained from the patient.

Embodiment 30. The method of any one of Embodiments 1 to 29, wherein a biological sample obtained from the patient has a normal level of carbohydrate antigen 19-9 (CA19-9).

Embodiment 31. The method of any one of Embodiments 1 to 29, wherein a biological sample obtained from the patient has an elevated level of carbohydrate antigen 19-9 (CA19-9).

Embodiment 32. The method of any one of Embodiments 1 to 31, wherein the biological sample obtained from the patient is Lewis antigen-negative.

Embodiment 33. The method of any one of Embodiments 1 to 32, wherein the biological sample is a blood sample.

Embodiment 34. The method of Embodiment 33, wherein the blood sample is a serum sample.

Embodiment 35. The method of Embodiment 33, wherein the blood sample is a plasma sample.

Embodiment 36. The method of any one of Embodiments 1 to 35, wherein the pancreatic cancer is Stage 1 pancreatic cancer or Stage 2 pancreatic cancer.

Embodiment 37. The method of any one of Embodiments 1 to 36, wherein the pancreatic cancer is Stage 1 pancreatic cancer.

Embodiment 38. The method of any one of Embodiments 1 to 36, wherein the pancreatic cancer is Stage 2 pancreatic cancer.

Embodiment 39. The method of any one of Embodiments 1 to 35, wherein the pancreatic cancer is Stage 3 pancreatic cancer or Stage 4 pancreatic cancer.

Embodiment 40. The method of any one of Embodiments 1 to 39, wherein the pancreatic cancer is pancreatic ductal adenocarcinoma.

Embodiment 41. The method of any one of Embodiments 1 to 40, wherein the patient has new-onset diabetes, has pre-existing diabetes, has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof.

Embodiment 42. The method of any one of Embodiments 1, 2, and 7-41 comprising administering to the patient the effective amount of the anti-cancer agent.

Embodiment 43. The method of Embodiment 42, wherein the anti-cancer agent comprises everolimus, erlotinib, olaparib, mitomycin, sunitinib, gemcitabine, 5-fluorouracil, irinotecan, oxaliplatin, paclitaxel, capecitabine, cisplatin, docetaxel, or a combination of two or more thereof.

Embodiment 44. The method of Embodiment 42, wherein the anti-cancer agent is a chemotherapeutic agent.

Embodiment 45. The method of Embodiment 44, wherein the chemotherapeutic agent comprises gemcitabine, 5-fluorouracil, irinotecan, oxaliplatin, paclitaxel, capecitabine, cisplatin, docetaxel, or a combination of two or more thereof.

Embodiment 46. The method of Embodiment 44, wherein the chemotherapeutic agent is an alkylating agent, an antimetabolite compound, an anthracycline compound, an antitumor antibiotic, a platinum compound, a topoisomerase inhibitor, a vinca alkaloid, a taxane compound, an epothilone compound, or a combination of two or more thereof.

Embodiment 47. The method of Embodiment 46, wherein the alkylating agent is carboplatin, chlorambucil, cyclophosphamide, melphalan, mechlorethamine, procarbazine, or thiotepa; the antimetabolite compound is azacitidine, capecitabine, cytarabine, gemcitabine, doxifluridine, hydroxyurea, methotrexate, pemetrexed, 6-thioguanine, 5-fluorouracil, or 6-mercaptopurine; the anthracycline compound is daunorubicin, doxorubicin, idarubicin, epirubicin, or mitoxantrone; the antitumor antibiotic is actinomycin, bleomycin, mitomycin, or valrubicin; the platinum compound is cisplatin or oxaliplatin; the topoisomerase inhibitor is irinotecan, topotecan, amsacrine, etoposide, teniposide, or eribulin; the vinca alkaloid is vincristine, vinblastine, vinorelbine, or vindesine; the taxane compound is paclitaxel or docetaxel; and the epothilone compound is epothilone, ixabepilone, patupilone, or sagopilone.

Embodiment 48. The method of any one of Embodiments 1, 2, and 7-47, comprising administering to the patient the effective amount of the radiation therapy.

Embodiment 49. The method of any one of Embodiments 1, 2, and 7-48, comprising administering to the patient image-based screening.

Embodiment 50. The method of Embodiment 49, wherein the image-based screening is computed tomography, magnetic resonance imaging, or endoscopic ultrasonography.

Embodiment 51. The method of any one of Embodiments 1, 2, and 7-50, comprising surgically removing all or a portion of the pancreas of the patient.

Embodiment 52. The method of any one of Embodiments 1-3 and 6-51, wherein the control is a patient or population of patients that do not have cancer.

Embodiment 53. The method of any one of Embodiments 1-3 and 6-51, wherein the control is a patient or population of patients that do not have pancreatic cancer.

Embodiment 54. A kit comprising reagents capable of detecting an expression level of RNA from a biological sample; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof.

Embodiment 55. The kit of Embodiment 54, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof.

Embodiment 56. The kit of Embodiment 54, wherein the RNA comprises cell-free miR-30c-5p, cell-free miR-142-3p, cell-free miR-340-5p, cell-free miR-335-5p, and cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p.

Embodiment 57. The kit of any one of Embodiments 54 to 56, wherein the biological sample is blood.

Embodiment 58. The method of any one of Embodiments 1-8, 10-22, and 30-53, wherein the RNA comprises cell-free let-7e-5p.

Embodiment 59. The method of any one of Embodiments 1-8, 10-22, 30-53, and 58, wherein the RNA comprises cell-free miR-26a-5p.

Embodiment 60. The method of any one of Embodiments 1-8, 10-22, 30-53, 58, and 59, wherein the RNA comprises cell-free miR-223-3p.

Embodiment 61. The method of any one of Embodiments 1-8, 10-22, 30-53, and 58-60, wherein the RNA comprises cell-free miR-340-3p.

Embodiment 62. The method of any one of Embodiments 1-8, 10-22, 30-53, and 58-61, wherein the RNA comprises exosomal miR-1260a.

Embodiment 63. The method of any one of Embodiments 1-8, 10-22, 30-53, and 58-62, wherein the RNA comprises exosomal miR-141-3p.

Embodiment 64. The method of any one of Embodiments 1-8, 10-22, 30-53, and 58-63, wherein the RNA comprises exosomal miR-143-3p.

Embodiment 65. The method of any one of Embodiments 1-8, 10-22, 30-53, and 58-64, wherein the RNA comprises exosomal miR-148a-3p.

Embodiment 66. The method of any one of Embodiments 1-8, 10-22, 30-53, and 58-65, wherein the RNA comprises exosomal miR-200c-3p.

Embodiment 67. The method of any one of Embodiments 1-8, 10-22, 30-53, and 58-66, wherein the RNA comprises exosomal miR-216a-5p.

Embodiment 68. The method of any one of Embodiments 1-8, 10-22, 30-53, and 58-67, wherein the RNA comprises exosomal miR-34a-5p.

Embodiment 69. The method of any one of Embodiments 1-7, 10-22, 30-53, and 58-68, wherein the RNA comprises cell-free let-7f-5p.

Embodiment 70. The method of any one of Embodiments 1-7, 10-22, 30-53, and 58-69, wherein the RNA comprises cell-free miR-369-3p.

Embodiment 71. The method of any one of Embodiments 1-7, 10-22, 30-53, and 58-70, wherein the RNA comprises cell-free miR-125a-5p.

Embodiment 72. The method of any one of Embodiments 1-7, 10-22, 30-53, and 58-71, wherein the RNA comprises cell-free miR-495-3p.

Embodiment 73. The method of any one of Embodiments 1-7, 10-22, 30-53, and 58-72, wherein the RNA comprises exosomal miR-375-3p.

Embodiment 74. The method of any one of Embodiments 1-7, 10-22, 30-53, and 58-73, wherein the RNA comprises exosomal miR-199a-5p.

### EXAMPLES

With recent advances in high-throughput molecular profiling technologies, the use of blood-based biomarkers for cancer diagnosis has gained significant momentum in the form of circulating proteins, DNA and various RNA molecules. Within the transcriptomic landscape, microRNAs (miRNAs) represent single-stranded RNAs that are 18-25 nucleotides long, that are involved in gene regulation and oncogenesis, and are frequently dysregulated in different cancers including PDAC. (Refs 22-23). Since miRNAs are resistant to nuclease-mediated degradation and its abundance in tissues, blood, and other body fluids due to their small size, they have emerged as promising candidates for liquid-biopsy based molecular biomarkers in human cancers. However, it is well-recognized that not only tumor cells, but multiple other sources, including apoptotic and immune cells release cell-free miRNAs (cf-miRNAs) in circulation. Thus, there is some debate in the field with regards to the potential heterogeneity associated with the origin of cf-miRNAs.

The recent discovery of exosomes, small membranous microvesicles about 40-140 nm in size, which inherit molecular signatures from their cells-of-origin, has brought a great degree of enthusiasm to the cancer biomarker arena. Initially, exosomes were thought to be involved in the disposal of cellular garbage, but recent data offers compelling evidence that they play an important role in cell-to-cell communication, through the transfer of their molecular cargo (e.g., proteins and nucleic acids) within the tumor microenvironment. Cancer cells, analogous to healthy cells, secrete exosomes and carry distinct pathogenic milieu. Exosomes excreted by cancer cells appear to possess specific exosomal cargos including miRNAs and offer abundant representation of tumor-derived miRNAs in systemic circulation; and hence provide an attractive paradigm for more specific detection of miRNA biomarkers in blood. Given that cf-miRNAs offer excellent sensitivity and exo-miRNAs are highly tissue-specific, a combination of the two could offer an optimal mix of sensitivity and specificity, an approach that is unique, merits attention and has not been previously explored for the early detection of pancreatic cancer.

In this present study, the inventors performed a systematic and comprehensive genome wide transcriptomic profiling of a large number of clinical specimens from patients with early-stage PDAC (stages I and II) and appropriate non-disease control subjects to discover a novel cell-free and exosomal miRNA signature that facilitates early detection of patients with earliest stages of PDAC. Following biomarker discovery, the inventors rigorously evaluated and validated the performance of this noninvasive circulating signature in multiple independent clinical cohorts to assess its diagnostic performance for the early detection of patients with PDAC.

### Summary of Examples

Background and Aims: Pancreatic ductal adenocarcinoma (PDAC) incidence is rising worldwide, and majority of patients present with an unresectable disease at initial diagnosis. Measurement of carbohydrate antigen 19-9 (CA19-9) levels lack adequate sensitivity and specificity for early detection; hence, there is an unmet need to develop alternate molecular diagnostic biomarkers for PDAC. Emerging evidence suggests that tumor-derived exosomal cargo, particularly miRNAs, offer an attractive platform for the development of cancer-specific biomarkers. Herein, we performed genome wide profiling in blood specimens to develop an exosome-based transcriptomic signature for noninvasive and early detection of PDAC. Methods: Small RNA-sequencing was performed in a cohort of 44 patients with an early-stage PDAC and 57 non-disease controls. Using machine-learning algorithms, we prioritized a panel of cell-free (cf) and exosomal (exo) miRNAs that discriminated PDAC patients from control subjects. Subsequently, we trained and validated the performance of the biomarkers in independent cohorts (n=191) using quantitative real time PCR (qRT-PCR) assays. Results: The sequencing analysis initially identified a panel 30 overexpressed miRNAs in PDAC. Subsequently using PCR assays, we reduced the panel to 13 markers (5 cf- and 8 exo-miRNAs), which successfully identified patients with all stages of PDAC (AUC=0.98 training cohort; AUC=0.93 validation cohort); but more importantly, was equally robust for the identification of early-stage PDAC (Stages 1 and 2; AUC=0.93). Furthermore, this transcriptomic signature successfully identified CA19-9 negative cases (<37 U/ml; AUC=0.96), when analyzed in combination with CA19-9 levels, significantly improved the overall diagnostic accuracy (AUC=0.99 vs. AUC=0.86 for CA19-9 alone). Conclusions: Shown herein is an exosome-based liquid-biopsy signature for the noninvasive and robust detection of patients with PDAC.

### Materials and Methods

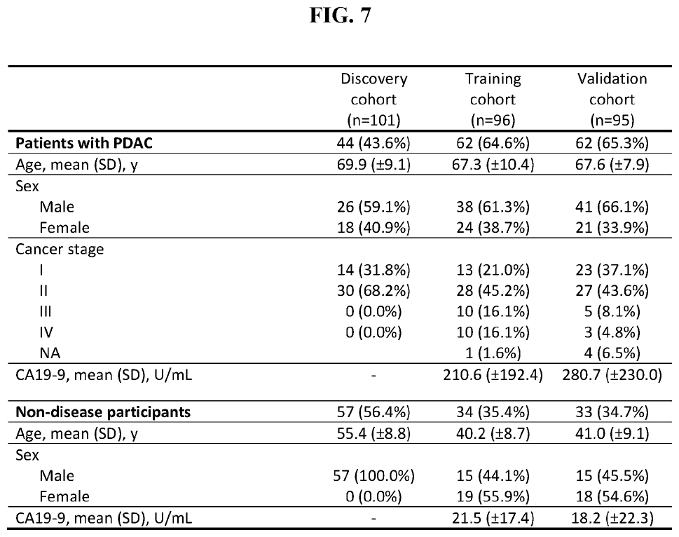

Patient cohorts. In this study, the inventors enrolled a total of 292 subjects (168 PDAC patients and 124 non-disease controls) which were segregated into a biomarker discovery cohort that was subjected to small RNA sequencing, as well as clinical training and validation cohorts. The detailed clinicopathological characteristics of all PDAC cases and non-disease controls are presented in FIG. 7. For the biomarker discovery phase, small RNA sequencing was performed in a total of 101 plasma and serum specimens which included 44 patients with early-stage PDAC (stages I and II) and 57 non-disease controls, who were enrolled at the Samsung Medical Center and Asan Medical Center, Seoul, Korea, between 2009 and 2017. In the clinical training and validation phases, qRT-PCR assays were performed to examine the expression levels of candidate cell-free and exosomal-miRNAs in 191 specimens from 124 patients with PDAC and 67 non-disease controls, who were enrolled at the Ochsner Clinic Foundation (New Orleans, LA, USA) and Nagoya University Hospital (Nagoya, Japan) between 2016 and 2020. To minimize any potential bias between these two patient populations, the two cohorts were combined and randomly divided into two cohorts (training cohort, n=96; validation cohort, n=95) for qRT-PCR based performance evaluation. The study was conducted in accordance with the Declaration of Helsinki. A written informed consent was obtained from all the subjects, and the study was approved by respective institutional review boards.

### Exosomal and Cell-Free RNA Extraction

To prepare libraries for small RNA sequencing, total exosomal RNA and cell-free RNA were isolated from 400 μL blood plasma or serum, using exoRNeasy Midi Kit and miRNeasy kit (Qiagen, Valencia, CA, USA), respectively. For qRT-PCR analysis, exosomes were first isolated from 200 μL plasma or serum was performed using Total Exosome Isolation Kit (Invitrogen, Waltham, MA, USA), followed by RNA extraction using miRNeasy Kit (Qiagen). Cell-free total RNA was isolated from 200 μL plasma or serum using miRNeasy Kit (Qiagen).

Small RNA Sequencing and Discovery Analysis for Identification of miRNA Candidates

Exosomal and cell-free RNA was prepared for library preparation by using the NEXTflex™ Small RNA-Seq Kit v3 (PerkinElmer, Waltham, MA, USA). Following size exclusion and quality assessment, the sequencing libraries were pooled, and paired-end sequencing was performed using an Illumina NovaSeq platform. The Cutadapt (v2.2) pipeline was used to trim the adapters and reads with low quality were removed. Next, the miRDeep2 module was used to align miRNA sequences (against miRBase release 22) and quantify miRNA expression. The miRNA abundance was calculated based on counts per million (CPM). The R package, limma, was used to perform differential expression analysis to identify miRNA candidates between early-stage PDAC patients and controls.

### Real-Time Quantitative Reverse Transcription Polymerase Chain Reaction

The cell-free and exosomal RNAs were first subjected to cDNA synthesis followed by LNA miRNA PCR assays using miRCURY LNA RT Kit (Qiagen). The expression of miRNAs was quantified by a SensiFAST™ SYBR Lo-ROX Kit (Bioline, London, UK) using QuantStudio 6 Flex Real-Time PCR System (Applied Biosystems, Foster City, CA, USA). The expression of miR-16-5p and miR103-3p were used as endogenous controls for data normalization. The expression of each miRNA was calculated using 2^{-ΔCt} method. Normalized expression values were further log₁₀ transformed.

### Measurement of CA19-9 Levels

The serum levels of CA19-9 were measured for all specimens analyzed for qRT-PCR based clinical training and validation by using the enzyme-linked immunosorbent assay (ELISA) kits from Alpha Diagnostic Intemational (San Antonio, TX, USA), as per manufacturer's instructions.

miRNA-mRNA Regulatory Network Analysis

miRNA-mRNA regulatory network analysis was performed based on miRNA-target interactions predicted by at least three programs using starBase (https://starbase.sysu.edu.cn/). For higher specificity, the network was further filtered to retain target genes (log 2-fold change >0.5 and adjusted P value <0.05) that were differentially expressed between PDAC and normal tissue samples using the GSE62452 dataset which included 69 pancreatic tumors and 61 adjacent non-tumor tissues. Furthermore, functional annotation was performed on the miRNA target genes in the network based on KEGG pathways and cancer hallmark gene sets in the MSigDB database using the “clusterProfiler” package. Subramanian et al, Proc Natl Acad Sci USA, 102:15545-50 (2005); Yu et al, Omics, 16:284-7 (2012).

### Statistical Analysis

Statistical analyses were performed using R (version 4.0.3, https://cran.r-project.org/), MedCalc Statistical Software version 20.009 (MedCalc Software Ltd, Ostend, Belgium) and GraphPad Prism 8 software (La Jolla, CA). Area under the curve values (AUCs) derived from the receiver operating characteristic (ROC) curves were calculated with confidence intervals (CIs) using the pROC package in R. All ROC curves presented in our results are represented along with 95% CI. The CI values were calculated by 2,000 bootstrap replicates. The optimal cutoff thresholds for the ROC curves were determined using Youden's index. The sensitivity, specificity, positive predictive value (PPV), negative predictive value (NPV), precision and accuracy of the miRNA signatures were calculated across all the cohorts using pROC package. A p value <0.05 was considered as statistically significant. The Wilcoxon test was used to compare risk scores from qRT-PCR experiments between different stages and healthy controls. Multivariate logistic regression was employed to derive a formula to predict risk for development of PDAC.

### Results

Genome wide profiling identifies a panel of cell-free and exosomal-miRNA biomarkers for the identification of patients with early-stage PDAC

The primary objective of the study was to identify clinically relevant cell-free miRNAs (cf-miRNAs) and exosomal miRNAs (exo-miRNAs) as biomarkers for the identification of patients with PDAC, but more importantly, for those with an early-stage disease. Towards these efforts, a systematic and comprehensive, unbiased, genome wide small RNA-sequencing based biomarker discovery approach was used in total plasma and exosomal RNA specimens from patients with early-stage PDAC (stages I and II) and non-disease controls. Following sequencing, rigorous bioinformatic and statistical analyses were performed to identify candidate miRNAs that were significantly and differentially expressed between PDAC patients (n=44) vs. non-disease controls (n=57). The non-disease controls subjects were defined as asymptomatic patients with normal abdominal CTs or negative screening endoscopies. The following criteria were used for prioritizing candidate miRNAs: for cf-miRNAs we used an AUC value of >0.7 and a log fold change of >1; and for exosomal miRNAs we used an AUC value of >0.8 and log fold change of >1. To ensure the robustness of identified candidate biomarkers, we performed 1000 time cross-validation and selected the markers that were most stable in such iterative analysis.

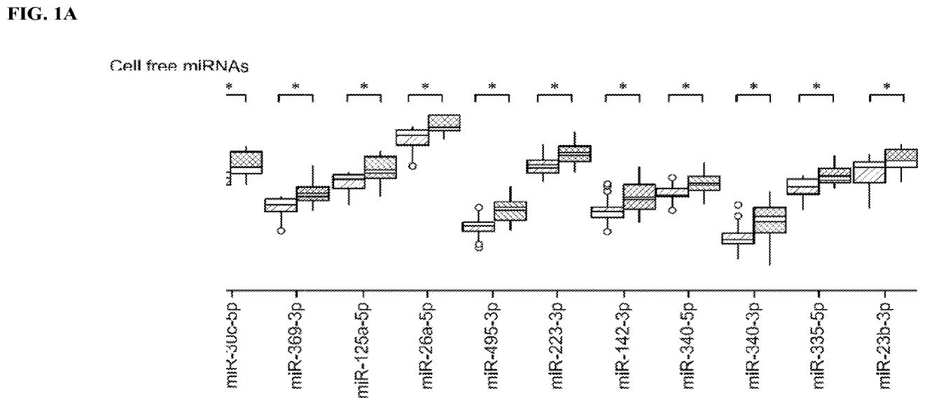

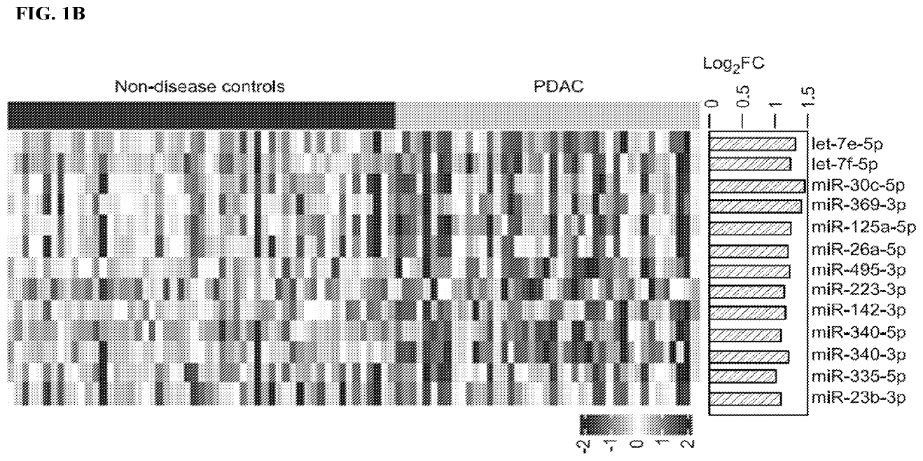

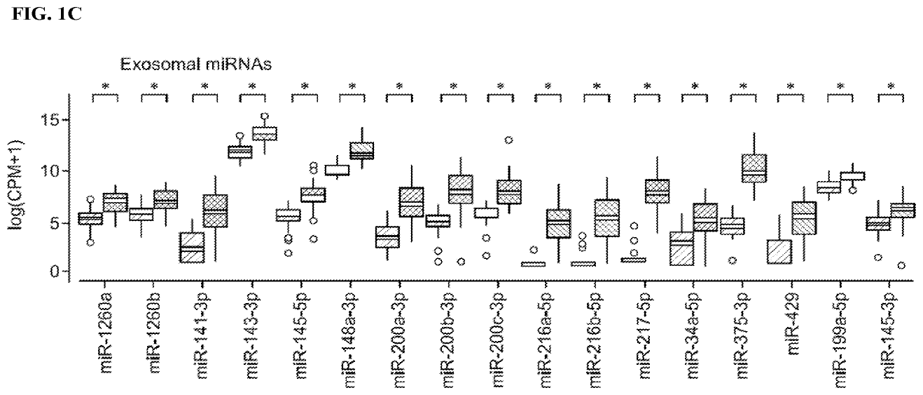

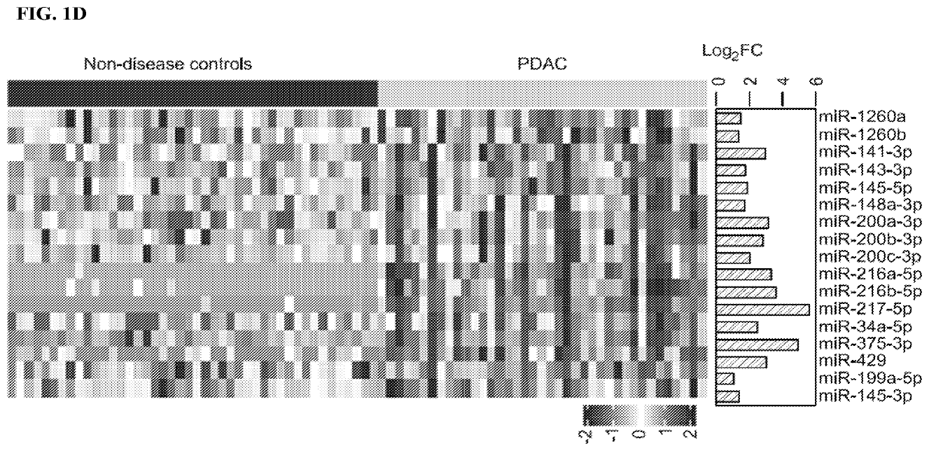

Bioinformatic and statistical analyses resulted in the identification of a panel of 13 cf-miRNAs (let-7e-5p, let-7f-5p, miR-30c-5p, miR-369-3p, miR-125a-5p, miR-26a-5p, miR-495-3p, miR-223-3p, miR-142-3p, miR-340-5p, miR-340-3p miR-335-5p, and miR-23b-3p) and 17 exo-miRNAs (miR-1260a, miR-1260b, miR-141-3p, miR-143-3p, miR-145-5p, miR-148a-3p, miR-200a-3p, miR-200b-3p, miR-200c-3p, miR-216a-5p, miR-216b-5p, miR-217-5p, miR-34a-5p, miR-375-3p, miR-429, miR-199a-5p, and miR-145-3p)—all of which were significantly upregulated in patients with stage I/II PDAC vs. non-disease controls (FIGS. 1A-1D). While these individual markers were quite robust, in order to evaluate the performance of these candidates as combinatorial panels, a logistic regression model was developed using the coefficients derived from each of the 13 cf- and 17 exo-miRNAs in this discovery cohort. It was interesting to observe that the predictive probabilities deduced from the logistic regression model demonstrated an excellent diagnostic potential for the two types of miRNA panels; where the cf-miRNA panel exhibited an AUC value of 0.96 (95% CI, 0.93-1.00; sensitivity of 92%, specificity of 93%, PPV of 94%, and NPV of 91%) and the exo-miRNA panel yielded an AUC of 1.00 (CI, 0.99-1.00; sensitivity of 98%, specificity of 100%, PPV of 100%, and NPV of 98%). However, more importantly, the performance of these individual marker panels was significantly enhanced when they were combined together as a combination signature (AUC, 1.00; 95% CI, 1.00-1.00; sensitivity of 100%, specificity of 100%, PPV of 100%, and NPV of 100%). In summary, these biomarker discovery findings supported the original hypothesis that while both cf- and exo-miRNA markers are quite robust, their cumulative analysis offers a superior combination of sensitivity and specificity for the robust identification of patients with early-stage PDAC.

Training of a miRNA-based risk prediction model for the early detection of patients with PDAC

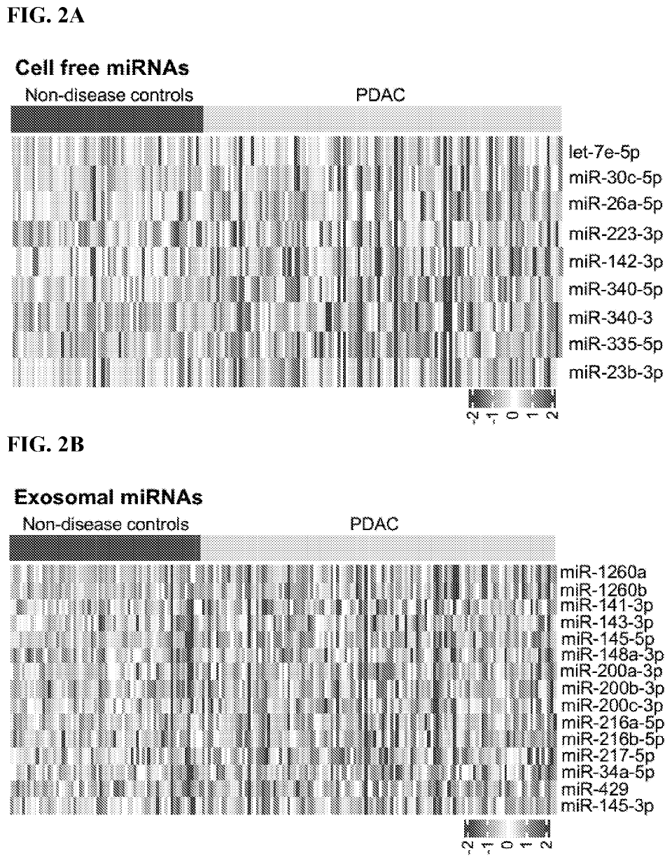

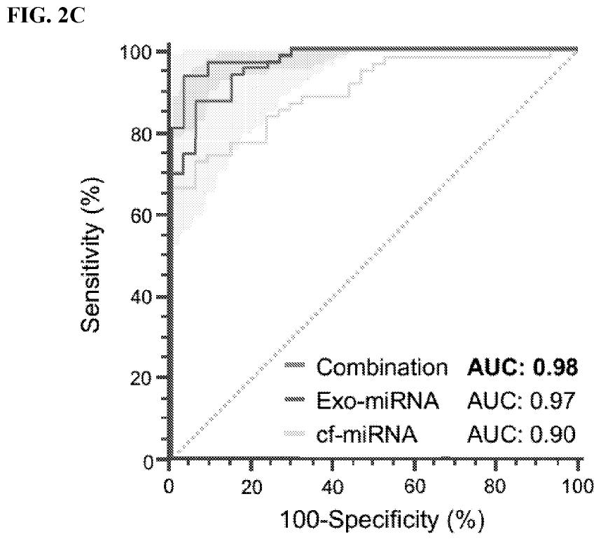

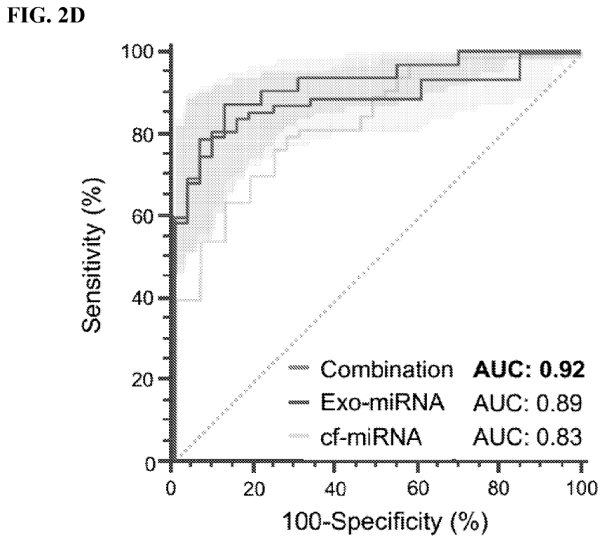

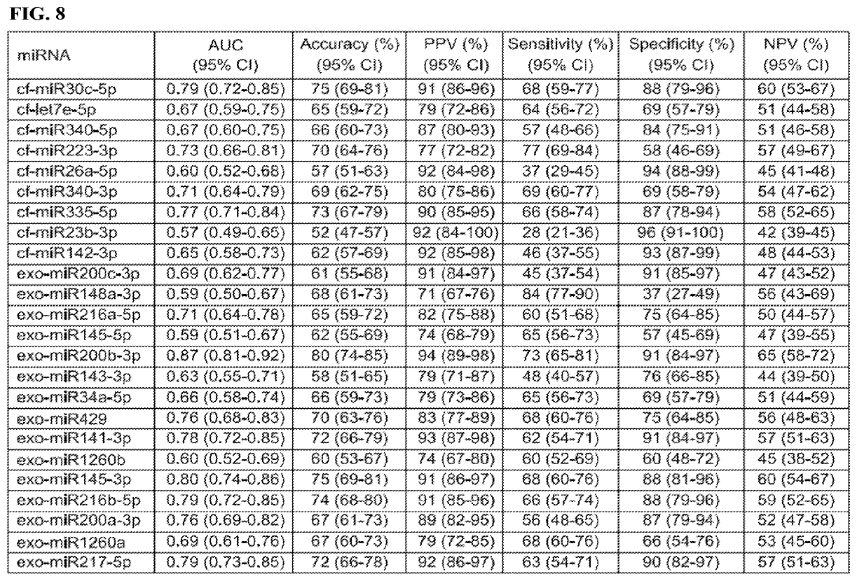

To establish a diagnostic assay for routine clinical implementation based upon the discovered miRNA biomarkers, qRT-PCR based assays were performed in blood specimens obtained from patients in independent clinical cohorts. It was discovered that even in qRT-PCR analysis, 9 of 13 cf-miRNAs (let-7e-5p, miR-30c-5p, miR-26a-5p, miR-223-3p, miR-142-3p, miR-340-5p, miR-340-3p, miR-335-5p, and miR-23b-3p) and 15 of 17 exo-miRNAs (miR-1260a, miR-1260b, miR-141-3p, miR-143-3p, miR-145-5p, miR-148a-3p, miR-200a-3p, miR-200b-3p, miR-200c-3p, miR-216a-5p, miR-216b-5p, miR-217-5p, miR-34a-5p, miR-429, and miR-145-3p) were readily detectable; underscoring the robustness of the biomarker discovery efforts. The significantly upregulated (log fold change >1 and p<0.05) cf- and exo-miRNA candidates between patients with PDAC and non-disease controls were represented as heat maps in FIGS. 2A-2B, respectively. The diagnostic performance of each miRNA candidate in terms of their AUC values, diagnostic accuracy, NPV, PPV, sensitivity and specificity are summarized in FIG. 8.

Based upon these findings, the 24 candidate miRNA biomarkers were subsequently included in a logistic regression analysis for training a risk-prediction model for the identification of patients with PDAC in a cohort of 62 PDAC patients and 34 non-disease controls. During this model development, the coefficients and constants derived from the logistic regression equation were applied to calculate risk scores for each of the markers within the cf- and exo-miRNA panels for their ability to diagnose any patient with PDAC, as follows: cf-miRNA panel-[(3.907893 X cf-miR-30c-5p)+(−0.13495 X cf-let7e)+(0.979111 X cf-miR340-5p)+(−0.01936 X cf-miR223-3p)+(−0.38275 X cf-miR-26a-5p)+(0.195893 X cf-miR340-3p)+(0.718554 X cf-miR335-5p)+(−1.75009 X cf-miR23b-3p)+(−1.3107 X cf-miR142-3p)+1.501128]; and exo-miRNA panel-[(−0.03638 X exo-miR200c-3p)+(1.087279 X exo-miR148a-3p)+(1.04184 X exo-miR216a-5p)+(−3.1777 X exo-miR145-5p)+(4.112727 X exo-miR200b-3p)+(0.057046 X exo-miR143-3p)+(0.118696 X exo-miR34a-5p)+(−0.92937 X exo-miR429)+(−0.57374 X exo-miR141-3p)+(−4.10024 X exo-miR1260b)+(2.42878 X exo-miR145-3p)+(0.962229 X exo-miR216b-5p)+(1.189049 X exo-miR200a-3p)+(1.691518 X exo-miR1260a)+(1.622576 X exo-miR217-5p)+2.981741]. While the performance of most individual markers was quite remarkable, the combined analysis of these markers within each panel was significantly superior in terms of their overall diagnostic accuracy (9 cf-miRNA panel AUC, 0.90; sensitivity, 73% and specificity, 94%; 15 exo-miRNA panel AUC, 0.97; sensitivity, 87% and specificity, 94%; FIGS. 2C and 5). Subsequently, the risk scores derived from cf- and exo-miRNA panels were combined together and the diagnostic potential of this combined transcriptomic signature was evaluated. In line with previous findings from the discovery cohort, the diagnostic performance of the trained model for this combined signature was significantly superior vis-a-vis individual panels, as this yielded an AUC value of 0.98 with a corresponding sensitivity of 94% and a specificity of 97% (FIGS. 2C and 5). Importantly, the overall diagnostic accuracy (95%), PPV (98%) and NPV (89%) of this combined signature was significantly superior to that of cf- and exo-miRNA panels individually, consistent with the findings from the biomarker discovery cohort. Taken together, this genome wide transcriptomic profiling efforts yielded clinically relevant miRNA biomarkers that will allow for successfully establishing and training a risk-prediction model for cf- and exo-miRNAs individually, as well as their combination, for the robust identification of patients with PDAC.

Successful validation of the circulating miRNA diagnostic signature in an independent cohort of patients with PDAC

Next, the diagnostic potential of the miRNA assay was investigated in an independent validation cohort of 62 PDAC patients and 33 non-disease controls by performing qRT-PCR based assays in plasma specimens. In this validation effort, the same logistic regression equation and the coefficients of each individual miRNAs and constants obtained from the training cohort model and calculated the risk scores was used. Consistent with the findings from the training cohort, the diagnostic potential for the cf-miRNA panel (AUC=0.83, Sensitivity=79% and Specificity=73%) and exo-miRNA panel (AUC=0.89, Sensitivity=79% and Specificity=94%;

FIGS. 2D and 5), was comparable even in this independent cohorts of PDAC patients and controls. Likewise, as was the case in the training cohort, the combined cf- and exo miRNA signature exhibited a superior diagnostic performance with an AUC value of 0.92, Sensitivity of 87%, Specificity of 88%, PPV of 93%, and NPV of 79%; FIGS. 2D and 5) in this validation cohort. Collectively, the diagnostic performance of the biomarker panels and the trained risk-prediction model in an independent cohort of patients with PDAC was successfully validated; highlighting that while individual cf and exo-miRNA panels are quite robust, the combined transcriptomic signature demonstrated a superior diagnostic performance for the identification of patients with PDAC.

Establishment of a clinically feasible signature using a reduced number of biomarkers for the noninvasive identification of patients with PDAC

In order to develop a clinically feasible and cost-effective assay that includes only the minimal number of markers required for maintaining the overall diagnostic performance of the cf- and exo-miRNAs, biomarker candidates using a systematic backward elimination approach were identified. This statistical strategy resulted in a reduced panel of 13 markers, which included 5 cf-miRNAs (miR-30c-5p, miR340-5p, miR335-5p, miR23b-3p and miR142-3p) and 8 exo-miRNA candidates (miR145-5p, miR200b-3p, miR429, miR1260b, miR145-3p, miR216b-5p, miR200a-3p and miR217-5p). The performance of these reduced marker panels was evaluated individually, and their combination, to discriminate PDAC patients from non-disease controls in both the clinical training and validation cohorts, as summarized in FIG. 5.

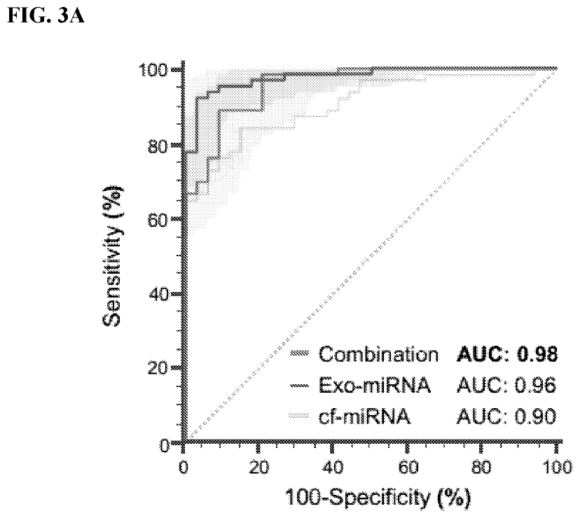

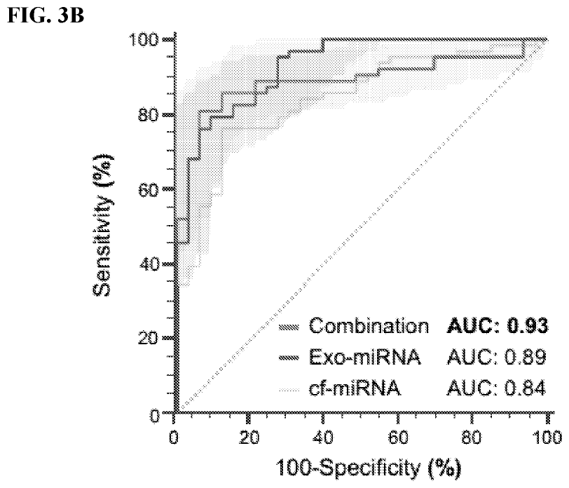

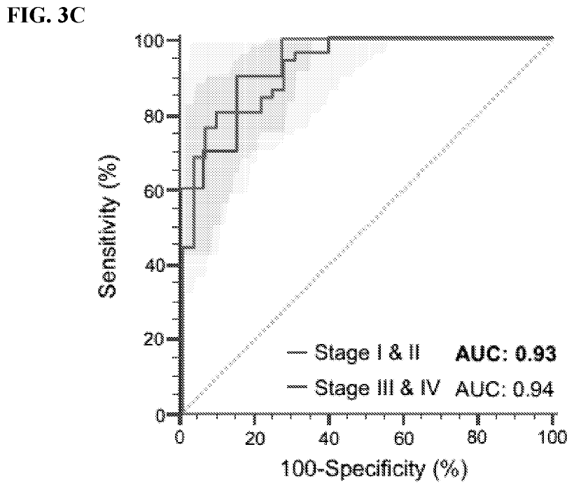

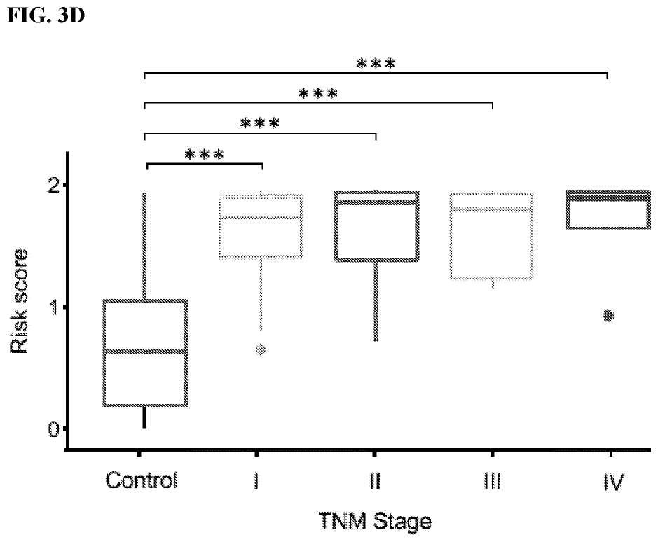

This reduced number of 13 markers was used in a logistic regression equation to re-calibrate the final risk-prediction model in the training cohort patients, as follows: cf-miRNA panel-[(3.8758 X cf-miR30c-5p)+(0.9970 X cf-miR340-5p)+(0.8286 X cf-miR335-5p)+(−1.9845 X cf-miR23b-3p)+(−1.4499 X cf-miR142-3p)+1.5448] and exo-miRNA panel-[(−2.9317 X exo-miR145-5p)+(3.2009 X exo-miR200b-3p)+(−1.2140 X exo-miR429)+(−1.3622 X exo-miR1260b)+(2.9393 X exo-miR145-3p)+(0.9949 X exo-miR216b)+(1.5168 X exo-miR200a-3p)+(1.4536 X exo-miR217-5p)+2.3454]. Using this recalibrated model, we observed that in the training cohort, the diagnostic AUC values for the cf- and exo-miRNA signatures were 0.90 and 0.96 respectively (FIGS. 3A and 5), which were consistent with the diagnostic performance of the larger pool of 24 markers trained and validated earlier (9 cf-miRNAs and 15 exo-miRNAs). Furthermore, the performance of the combined transcriptomic signature using this reduced panel of biomarkers exhibited an improved overall diagnostic performance with an AUC of 0.98, accuracy of 94%, PPV of 98%, sensitivity of 92% and a specificity of 97% (FIGS. 3A and 5).

Likewise, when the re-trained risk-prediction model was applied to the independent clinical validation cohort, the combined transcriptomic signature offered superior diagnostic performance with an AUC value of 0.93 vs. the cf-miRNA panel (AUC, 0.84) and exo-miRNA panel (AUC, 0.89; FIG. 3B). Taken together, these results demonstrate that the reduced transcriptomic panel is highly robust and provides a clinically attractive and inexpensive assay for the early detection of patients with PDAC.

The optimized transcriptomic signature performs robustly even for the identification of patients with the early-stage PDAC

While previous studies have attempted to develop early detection biomarkers in PDAC patients, a majority of these studies have not focused in evaluating the performance of these assays in earliest disease stages, which is as essential criterion for improving the prognosis of patients suffering from this fatal malignancy. Therefore, in the present study, the performance of the optimized 13 miRNA-based transcriptomic assay was evaluated for the diagnosis of patients with early-stage PDAC (stages I and II). The clinical validation cohort performed remarkably well for the identification of patients with stage III and IV cancers (AUC 0.94; Sensitivity, 90%; Specificity, 85%; PPV, 64%; NPV, 97% and Accuracy 86%), but the optimized combination transcriptomic assay performed remarkably well for the identification of patients with stage I and II cancers (AUC, 0.93; Sensitivity, 80%; Specificity, 91%; PPV, 93%; NPV, 76% and Accuracy 84%; FIG. 3C).

Furthermore, when the performance of the assay was compared in individual disease stages of PDAC patients vs. non-disease control subjects, the transcriptomic signature exhibited high risk score in all stages of patients with PDAC even in the early-stage lesions in the validation cohort (Mean risk score: non-disease controls, 0.68; stage I, 1.62; stage II, 1.67; stage III, 1.64; stage IV, 1.73; p<0.001; FIG. 3D). These results once again highlight the clinical significance of this exosome-based transcriptomic assay, which performs equally robustly in earliest stages of PDAC, presenting an ideal option for non-invasive identification of patients with this malignancy.

Exosome-based transcriptomic signature and CA19-9 levels in blood significantly improve diagnostic accuracy for PDAC

In routine clinical practice, CA19-9 is the only available blood-based biomarker for the management of patients with PDAC; however, it lacks sensitivity and specificity required for the early detection of patients in general population. Therefore, the inventors sought to explore whether combining the transcriptomic signature together with the CA19-9 glycoprotein could further improve the diagnostic performance of the assay in clinical settings. Accordingly, CA19-9 levels in all clinical specimens were measured, and the diagnostic performance of CA19-9 by itself, and also in conjunction with the transcriptomic signature described herein, was evaluated. The CA19-9 by itself yielded an AUC value of 0.88, in all stages of PDAC patients. When combined with the optimized signature described herein, a significant improvement in the overall diagnostic performance resulted, as evidenced by a superior AUC value of 0.99 (FIG. 4A). Even more noteworthy was the finding that this diagnostic performance was equally remarkable even when the results were analyzed in early-stage PDAC patients with stage I and II lesions (AUC for CA19-9, 0.86 vs. AUC, 0.99 in combination with transcriptomic signature; FIG. 4B).

The inventors observed that 22 PDAC patients had CA19-9 levels lower than 37 U/ml, a cut-off threshold below which individuals are generally deemed negative for PDAC in clinical settings. However, it is well known that 15-25% of pancreatic cancer patients present with a normal CA19-9, and about 5-10% of the general population is Lewis antigen-negative with no or low secretion of CA19-9. These clinical challenges prompted the inventors to perform a subgroup analysis for evaluating the performance of the transcriptomic signature in a subgroup of 81 subjects (22 PDAC and 59 non-disease controls) who presented with CA19-9 levels lower than 37 U/ml. Consistent with the previous findings, the assay described herein exhibited an excellent diagnostic performance with AUC value of 0.96, sensitivity of 91% and a specificity of 90%, whereas CA19-9 exhibited poor performance with an AUC value of 0.63, sensitivity of 68% and a specificity of 64% in this sub group (FIG. 4C). Interestingly, the miRNA transcriptomic signature described herein could identify 20 out of 22 patients with PDAC (91%) who were considered CA19-9 negative with the CA19-9 levels lower than 37 U/ml. Collectively, these results demonstrate that the miRNA transcriptomic signature described herein provides for a vastly improved diagnostic pancreatic cancer screening that can be complemented with CA19-9.

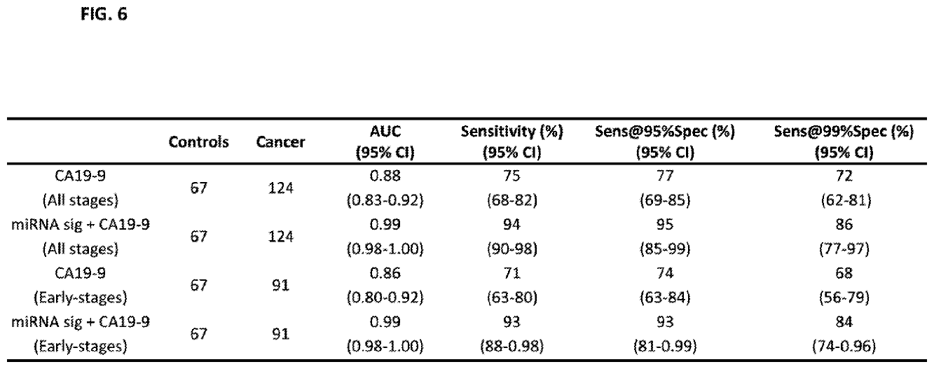

Next, the inventors explored the diagnostic performance of CA19-9 and the transcriptomic signature in combination with CA19-9 levels, after locking down the specificity of the assay at 95% and 99%, respectively. In the case of CA19-9 levels alone at a fixed specificity of 95% and 99%, a significantly lower sensitivity of 77% and 72% in all stages of PDAC patients and 74% and 68% in early-stage of patients with PDAC, respectively, was observed. However, combining the transcriptomic signature described herein together with CA19-9 levels yielded a remarkably high sensitivity of 95% and 86% respectively, for all stages of PDAC patients, and a sensitivity of 93% and 84% in patients even in the early-stage PDAC patients (FIG. 6). These findings once again highlight that while the exosome-based transcriptomic assay is quite robust on its own, when combined together with CA19-9 levels, it results in a significant improvement in the overall diagnostic accuracy, highlighting its ability to provide early detection of patients with PDAC.

An exosome-based liquid biopsy assay offers a significant benefit vs. current treatment approaches used in the clinic for the early detection of patients with PDAC

In current clinical practice, diagnosis of PDAC patients were achieved by computed tomography or an invasive biopsy followed by surgery if it is resectable. Accordingly, false positive or false negative cases based on current clinical practice would be detrimental to subjects undergoing this screening. Thus, the clinical usefulness of screening strategies should be estimated by the trade-off between the harm and diagnosis. To further examine the clinical significance of the transcriptomic assay described herein, a decision curve analysis (DCA) and calibration curve analysis were conducted. As shown in FIG. 4D, the X-axis represents the threshold probability for diagnosis of PDAC and the Y-axis represents the net benefit achieved. The DCA curve revealed that the exosome-based transcriptomic signature achieved a higher net benefit across most ranges of threshold probability in comparison to diagnosing all PDAC patients or none of the patients. (FIG. 4D). For instance, at threshold probability of 0.50, the transcriptomic signature exhibited a significantly higher net benefit of 0.45 for the diagnosis of PDAC vs. diagnosing all PDAC patients based on the strategy of intervention for all cases with a significantly lower net benefit of approximately 0.30. These findings indicate that the transcriptomic signature offers markedly higher clinical benefit compared to intervention for all cases or none of the cases, in terms of the viewpoint of the avoidance of harm and misdiagnosis. In addition, the calibration plots showed a good agreement between the observed vs. predicted probability across all ranges (FIG. 4E). A slight underestimation for diagnosis of PDAC was observed when the predicted probabilities were between 0.50-0.75 range. As the risk score increases from 0 to 1, the proportion of cancer patients also increases, highlighting that the exosome-based liquid biopsy signature exhibited robust diagnostic accuracy for identification of patients with PDAC.

### DISCUSSION

Pancreatic ductal adenocarcinoma (PDAC) is a lethal malignancy, with a rising incidence, but lacks adequate approaches for its early detection. Accumulating evidence indicates that miRNA-based liquid biopsy assays offer a promising strategy for the early detection of multiple human cancers including pancreatic cancer—primarily, due to their small size, resistance to nuclease-mediated degradation and their abundant and tissue-specific presence in various bodily fluids. However, there is some debate whether cell-free (cf)-miRNAs, which are often released in systemic circulation from multiple cellular sources, represent adequate diagnostic specificity for cancer detection. Recently, there has been burgeoning interest in studying tumor-derived exosomes and their cargo which contains various proteins and nucleic acids, particularly specific miRNAs (exo-miRNAs), which might provide an additional measure of specificity required for improving their overall diagnostic accuracy in cancer patients. Herein, it was hypothesized that a combination of cf- and exo-miRNAs might offer an attractive approach that maximizes the sensitivity and specificity of miRNA signatures in a liquid biopsy assay for cancer diagnosis. Accordingly, in the present study, a systematic and comprehensive biomarker discovery approach was used by performing small RNA sequencing in patients with early-stage PDAC (stages I and II) and non-disease control subjects, to discover and subsequently validate a transcriptomic signature for noninvasive identification of patients with PDAC.

A previous study has explored the diagnostic performance of a cf-miRNA signature for detection of PDAC and observed that miRNA panel could diagnose patients with PDAC with an AUC value of 0.93 to 0.97 in the training cohort and 0.81 to 0.83 in the validation cohort. (Ref 32). In line with this previous study, the cf-miRNA panel described herein also demonstrated robust performance for the detection of PDAC with an AUC value of 0.90 in the training cohort and 0.83 in the validation cohort. However, there were several potential challenges associated with this previous study including the lack of a genome wide biomarker discovery effort, inclusion of PDAC specimens mostly from advanced stage patients, and the inclusion of the patients with similar race and ethnicity. To mitigate these potential challenges in the present study the inventors performed small RNA sequencing by specifically analyzing only early-stage of PDAC specimens (stages I and II) for the discovery of candidate miRNAs, as well as trained and validated the performance of the biomarkers in multiple, independent clinical cohorts of patients from diverse populations of PDAC patients—with the discovery cohort specimens from Korea and training and validation specimens from Japan and USA, respectively.

While the cf- and exo-miRNA biomarker panels described herein performed remarkable well on their own, it was observed that a combined miRNA signature demonstrated a superior diagnostic performance for its ability to identify PDAC patients with an AUC value of 0.98 in the training cohort and 0.92 in the validation cohort. This novel strategy overcomes the limitations associated with the analysis of conventional analysis of cf-miRNAs. (Ref 32). Furthermore, in order to develop a clinically feasible and cost-effective assay that includes only the minimal number of markers required for maintaining the overall diagnostic performance of the cf- and exo-miRNAs, the inventors next prioritized biomarker candidates using a systematic backward elimination approach which led to identification of a 13 miRNA signature (5 cf- and 8 exo-miRNAs), which performed equally robustly with an AUC of 0.97 and 0.92 in the clinical training and validation cohorts, respectively. Even more importantly, this reduced and optimized exosome-based transcriptomic signature exhibited an impressive diagnostic accuracy (AUC, 0.96) for the identification of patients with early-stage (stages I and II) PDAC patients.

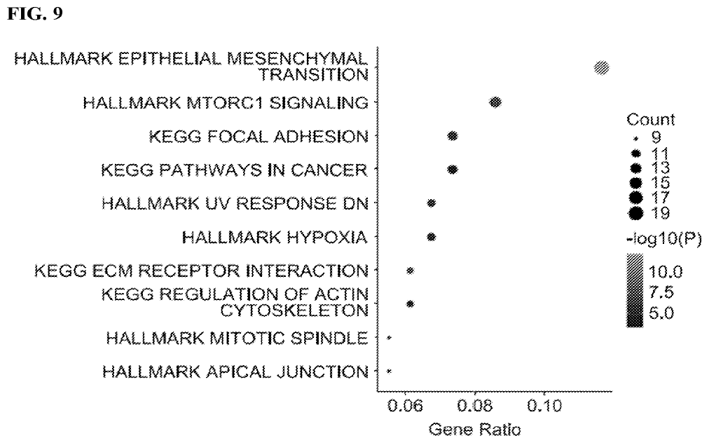

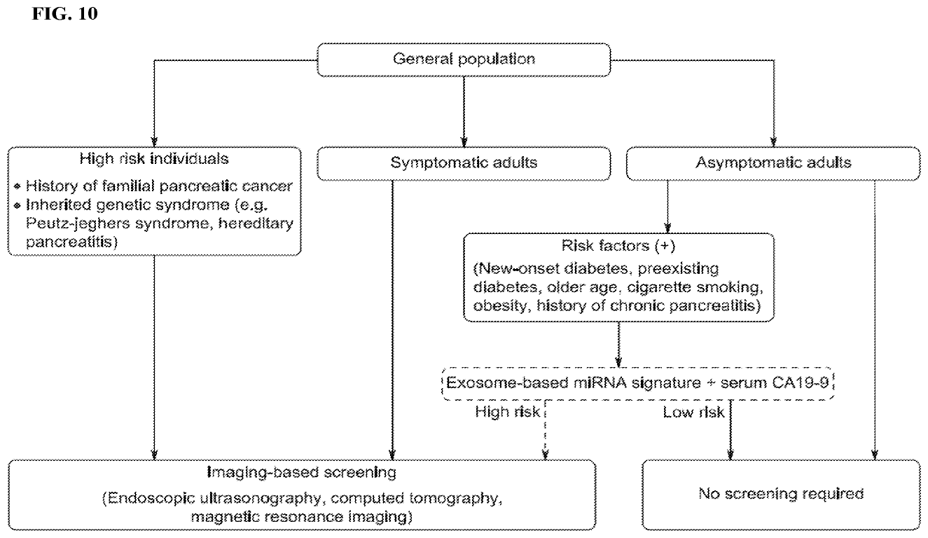

To better understand the functional relevance of these candidate miRNAs, a miRNA-mRNA regulatory network was constructed based on miRNA-target interactions predicted by StarBase. The network was further filtered to retain 165 target genes based on their log₂-fold change >0.5 and adjusted P<0.05 that were differentially expressed between PDAC and normal tissue samples using the GSE62452 dataset. More than half of the miRNA targets (54.5%) were cancer-related genes annotated in the cancer hallmark gene sets in the MSigDB database (version 7.0). Furthermore, functional annotation based on KEGG pathways and cancer hallmark gene sets in the MSigDB database showed that the miRNA target genes were significantly enriched in cancer-related signaling pathways such as epithelial mesenchymal transition, pathways in cancer, mTORC1 signaling etc. highlighting their strong functional relevance in pathogenesis of PDAC (FIG. 9).

In this study, the performance of the combined miRNA signature with respect to conventional serological tumor marker CA19-9 was also evaluated. Several previous studies have demonstrated that CA19-9 has a diagnostic potential for the detection of PDAC. However, for the early diagnosis of patients with pancreatic cancer, CA19-9 lacks sufficient diagnostic performance to be used as definitive molecular biomarker. In ideal scenario, sensitivity is particularly important for cancer screening because the screening strategy should provide maximum sensitivity to minimize the failure of identifying the disease. In the present study, CA19-9 alone had only 71% of sensitivity for the diagnosis of early-stage PDAC with false-negative results observed in PDAC specimens. On the other hand, when the transcriptomic signature described herein was combined with the CA19-9 level, the final diagnostic model showed a superior diagnostic performance with a sensitivity of 95% which was significantly higher as compared to CA19-9 alone. Moreover, when the specificity was fixed at 95% or 99%, the miRNA signature combined with CA19-9 could successfully maintain high sensitivity. These findings highlighted that the transcriptomic signature described herein significantly improves the diagnostic performance for pancreatic cancer screening that can be complemented with CA19-9.

Although CA19-9 is currently the most important biomarker for pancreatic cancer, 15-25% of pancreatic cancer patients present with a normal CA19-9. Moreover, approximately 5-10% of the population are Lewis antigen-negative with no or low secretion of CA19-9. These findings have been considered as one of the major weaknesses for CA19-9 to be used as a diagnostic biomarker for PDAC patients. Considering the limitation of CA19-9 in pancreatic cancer detection, the development of biomarkers that can complement CA19-9 in the management of Lewis negative pancreatic cancer is urgently needed. In the present study, the inventors observed that 22 PDAC patients had CA19-9 levels lower than normal limit (37 U/ml), these patients are generally considered negative for PDAC in clinic. When the subset analysis in patients with normal CA19-9 levels was performed, the miRNA signature could distinguish 91% of patients with PDAC from this cohort. This result highlighted that the miRNA signature described herein is a novel diagnostic strategy for PDAC patients with a normal CA19-9 or Lewis antigen-negative. Together, the transcriptomic signature was able to correctly classify them as PDAC, once again underscoring its importance for use in pancreatic cancer screening.

In conclusion, using a systematic and comprehensive biomarker discovery followed by successful clinical validation, the study described herein provides evidence of the clinical significance of an exosome-based transcriptomic signature for a noninvasive, liquid biopsy assay for the early detection of patients with pancreatic cancer.

### REFERENCES

1. Ryan et al, N Engl J Med 2014; 371:1039-49.
2. Kleeff et al, Nat Rev Dis Primers 2016; 2:16022.
3. Mizrahi et al, The Lancet 2020; 395:2008-2020.
4. Sung et al, CA Cancer J Clin 2021; 71:209-249.
5. Rahib et al, Cancer Res 2014; 74:2913-21.
6. Vincent et al, The Lancet 2011; 378:607-620.
7. Uesaka et al, The Lancet 2016; 388:248-257.
8. Conroy et al, N Engl J Med 2018; 379:2395-2406.
9. Conroy et al, N Engl J Med 2011; 364:1817-25.
10. Von Hoff et al, N Engl J Med 2013; 369:1691-703.
11. Golan et al, N Engl J Med 2019; 381:317-327.
12. Fahrmann et al, Gastroenterology 2021; 160:1373-1383 e6.
13. Majumder et al, Clin Cancer Res 2021; 27:2523-2532.
14. Gui et al, Clin Exp Med 2014; 14:225-33.
15. Tempero et al, Cancer Res 1987; 47:5501-3.
16. Martin et al, Med Oncol 2012; 29:3101-7.
17. Luo et al, Ann Surg 2017; 265:800-805.
18. Cescon et al, Nat Cancer 2020; 1:276-290.
19. Pantel et al, Nat Rev Clin Oncol 2019; 16:409-424.
20. Crowley et al, Nat Rev Clin Oncol 2013; 10:472-84.
21. Kilgour et al, Cancer Cell 2020; 37:485-495.
22. Toiyama Y, Okugawa Y, Fleshman J, et al. MicroRNAs as potential liquid biopsy biomarkers in colorectal cancer: A systematic review. Biochim Biophys Acta Rev Cancer 2018; 1870:274-282.
23. Shigeyasu K, Toden S, Zumwalt T J, et al. Emerging Role of MicroRNAs as Liquid Biopsy Biomarkers in Gastrointestinal Cancers. Clin Cancer Res 2017; 23:2391-2399.
24. Bloomston M, Frankel W L, Petrocca F, et al. MicroRNA expression patterns to differentiate pancreatic adenocarcinoma from normal pancreas and chronic pancreatitis. Jama 2007; 297:1901-8.
25. Nishiwada S, Cui Y, Sho M, et al. Transcriptomic Profiling Identifies an Exosomal microRNA Signature for Predicting Recurrence Following Surgery in Patients with Pancreatic Ductal Adenocarcinoma. Ann Surg 2021.
26. Jin X, Chen Y, Chen H, et al. Evaluation of Tumor-Derived Exosomal miRNA as Potential Diagnostic Biomarkers for Early-Stage Non-Small Cell Lung Cancer Using Next-Generation Sequencing. Clin Cancer Res 2017; 23:5311-5319.
27. Yu W, Hurley J, Roberts D, et al. Exosome-based liquid biopsies in cancer: opportunities and challenges. Ann Oncol 2021; 32:466-477.
28. Krug A K, Enderle D, Karlovich C, et al. Improved EGFR mutation detection using combined exosomal RNA and circulating tumor DNA in NSCLC patient plasma. Annals of Oncology 2018; 29:700-706.
29. San Lucas F A, Allenson K, Bernard V, et al. Minimally invasive genomic and transcriptomic profiling of visceral cancers by next-generation sequencing of circulating exosomes. Ann Oncol 2016; 27:635-41.
30. Ingenito F, Roscigno G, Affinito A, et al. The Role of Exo-miRNAs in Cancer: A Focus on Therapeutic and Diagnostic Applications. Int J Mol Sci 2019; 20.
31. Humphris et al, Ann Oncol 2012; 23:1713-22.
32. Schultz N A, Dehlendorff C, Jensen B V, et al. MicroRNA biomarkers in whole blood for detection of pancreatic cancer. JAMA 2014; 311:392-404.
33. So J B Y, Kapoor R, Zhu F, et al. Development and validation of a serum microRNA biomarker panel for detecting gastric cancer in a high-risk population. Gut 2021; 70:829-837.
34. O'Brien et al, Clin Cancer Res 2015; 21:622-31.

1. A method of detecting an RNA biomarker in a patient having, or suspected of having, pancreatic cancer, the method comprising detecting an elevated expression level, relative to a control, of an RNA in a biological sample obtained from the patient, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof.

2. A method of treating pancreatic cancer in a patient in need thereof, the method comprising administering to the patient an effective amount of an anti-cancer agent, administering to the patient an effective amount of radiation therapy, administering to the patient image-based screening, surgically removing all or a portion of the pancreas of the patient, or a combination of two or more thereof, wherein a biological sample obtained from the patient comprises an elevated expression level, relative to a control, of an RNA; wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof.

3. A method of treating pancreatic cancer in a patient in need thereof, the method comprising:
(i) detecting an elevated expression level, relative to a control, of an RNA in a biological sample obtained from a patient, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, cell-free let-7f-5p, cell-free miR-369-3p, cell-free miR-125a-5p, cell-free miR-495-3p, exosomal miR-375-3p, exosomal miR-199a-5p, or a combination of two or more thereof; and
(ii) administering to the patient an effective amount of an anti-cancer agent, administering to the patient an effective amount of radiation therapy, administering to the patient image-based screening, surgically removing all or a portion of the pancreas of the patient, or a combination of two or more thereof.

4-6. (canceled)

7. The method of claim 1, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, cell-free let-7e-5p, cell-free miR-26a-5p, cell-free miR-223-3p, cell-free miR-340-3p, exosomal miR-1260a, exosomal miR-141-3p, exosomal miR-143-3p, exosomal miR-148a-3p, exosomal miR-200c-3p, exosomal miR-216a-5p, exosomal miR-34a-5p, or a combination of two or more thereof.

8. The method of claim 1, wherein the RNA comprises cell-free miR-30c-5p, cell-free miR-142-3p, cell-free miR-340-5p, cell-free miR-335-5p, cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, exosomal miR-145-3p, or a combination of two or more thereof.

9. The method of claim 1, wherein the RNA comprises cell-free miR-30c-5p, cell-free miR-142-3p, cell-free miR-340-5p, cell-free miR-335-5p, and cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p.

10. The method of claim 1, wherein the RNA consists of cell-free miR-30c-5p, cell-free miR-142-3p, cell-free miR-340-5p, cell-free miR-335-5p, and cell-free miR-23b-3p, exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p.

11. The method of claim 1, further comprising detecting a level of carbohydrate antigen 19-9 (C-A-9-9) in the biological sample obtained from the patient.

12. The method of claim 1, wherein the biological sample obtained from the patient is Lewis antigen-negative.

13. The method of claim 1, wherein the biological sample is a blood sample.

14. The method of claim 1, wherein the patient has new-onset diabetes, has pre-existing diabetes, has obesity, has a rising carbohydrate antigen 19-9 level, has a family history of pancreatic cancer, has a history of pancreatitis, is ≥40 years old, smokes cigarettes, has a history of smoking cigarettes, or a combination of two or more thereof.

15. The method of claim 1, further comprising administering to the patient the effective amount of the anti-cancer agent.

16. The method of claim 15, wherein the anti-cancer agent comprises everolimus, erlotinib, olaparib, mitomycin, sunitinib, gemcitabine, 5-fluorouracil, irinotecan, oxaliplatin, paclitaxel, capecitabine, cisplatin, docetaxel, or a combination of two or more thereof.

17. The method of claim 15, wherein the anti-cancer agent is a chemotherapeutic agent.

18. The method of claim 17, wherein the chemotherapeutic agent comprises gemcitabine, 5-fluorouracil, irinotecan, oxaliplatin, paclitaxel, capecitabine, cisplatin, docetaxel, or a combination of two or more thereof.

19. The method of claim 17, wherein the chemotherapeutic agent is an alkylating agent, an antimetabolite compound, an anthracycline compound, an antitumor antibiotic, a platinum compound, a topoisomerase inhibitor, a vinca alkaloid, a taxane compound, an epothilone compound, or a combination of two or more thereof.

20. (canceled)

21. The method of claim 1, wherein the pancreatic cancer is Stage 1 pancreatic cancer or Stage 2 pancreatic cancer.

22. The method of claim 1, wherein the control is a non-diseased control.

23. The method of claim 1, wherein the RNA comprises cell-free miR-30c-5p, cell-free miR-142-3p, cell-free miR-340-5p, cell-free miR-335-5p, and cell-free miR-23b-3p.

24. The method of claim 1, wherein the RNA comprises:
(a) exosomal miR-1260b, exosomal miR-145-5p, exosomal miR-200a-3p, exosomal miR-200b-3p, exosomal miR-429, and exosomal miR-145-3p, or
(b) exosomal miR-1260b, exosomal miR-145-5p, exosomal miR-200a-3p, exosomal miR-200b-3p, exosomal miR-216b-5p, exosomal miR-217-5p, exosomal miR-429, and exosomal miR-145-3p.
